(19) United States
(12) Patent Application Publication
(10) Pub. No.: US 2025/0033056 A1
(43) Pub. Date: Jan. 30, 2025

(54) MICROWELL ARRAY CHIP, USE METHOD THEREFOR, AND DETECTION APPARATUS

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Ding DING, Beijing (CN); Jian XU, Beijing (CN); Lin DENG, Beijing (CN); Zhukai LIU, Beijing (CN)

(73) Assignees: BEIJING BOE TECHNOLOGY DEVELOPMENT CO., LTD., Beijing (CN); BOE TECHNOLOGY GROUP CO., LTD., Beijing (CN)

(21) Appl. No.: 18/713,233

(22) PCT Filed: Apr. 20, 2022
(86) PCT No.: PCT/CN2022/087880

§ 371 Date: May 24, 2024

(51) Int. Cl. B01L 3/00

(52) U.S. Cl. CPC B01L 3/50857; B01L 2300/0829; B01L 2300/0851; B01L 2300/0887; B01L 2300/161

(57) ABSTRACT

A microwell array chip, a use method therefor, and a detection apparatus. The microwell array chip includes a microwell array substrate, and the microwell array substrate includes n reaction chambers, and an idle region; an array of the n reaction chambers is disposed in the microwell array substrate, and the idle region is disposed around the n reaction chambers; each reaction chamber is configured to accommodate a sample to be tested, and the shape of an orthographic projection of the reaction chamber on a first reference plane where the first main surface is located is of a regular N-gon; the area of the idle region is divided into n′ virtual units, and the shape of the orthographic projection of each virtual unit on the first reference plane is the same as the shape of the orthographic projection of the reaction chamber on the first reference plane.

### TECHNICAL FIELD

At least one embodiment of the present disclosure relates to a microwell array chip, an using method thereof and a detection device.

### BACKGROUND

Polymerase chain reaction (PCR) is a molecular biological technique that can amplify DNA fragments in vitro, digital PCR technology (dPCR) is a new technology for absolute quantification of nucleic acid molecules, the dPCR technology does not depend on the standard curve and reference samples, and can directly detect a copy number of the target molecule without setting up a comparison. The principle of digital PCR technology is to distribute low-template reagents into a large number of microwells, after statistical analysis, there is no or only one target molecule in most of the microwells, after the amplification is completed, target molecules are counted by an optical detection module, which is an initial template amount. Compared with traditional quantitative PCR, the digital PCR technology has higher sensitivity, specificity, high tolerance and accuracy, this technology has been widely used in the detection of extremely small amounts of nucleic acid samples, CNV analysis and gene expression detection of complex samples.

A microwell array chip is a substrate including an array formed by a plurality of microwells, each of the microwells can be used as a small test tube to detect or select a specific compound among many compounds. Therefore, the digital PCR technology can use a microwell array chip for detection, by squeezing reagents into the channel of the chip and distributing the reagents into a large number of microwells.

### SUMMARY

Embodiments of the present disclosure provide a microwell array chip, a using method thereof, and a detection device. The microwell array chip includes a microwell array substrate, the microwell array substrate includes a first main surface and a second main surface that are oppositely arranged, n reaction chambers and a virtual idle region; the n reaction chambers are arranged in an array in the microwell array substrate, and the virtual idle region is arranged around the n reaction chambers; the n reaction chamber is configured to accommodate the sample to be tested, and an area of the virtual idle region is divided into n′ virtual units, a shape of an orthographic projection of each of the reaction chambers on the first reference plane where the first main surface is located is a regular N-polygon, a shape of an orthographic projection of each of the virtual units on the first reference plane is the same as the shape of the orthographic projection of each of the reaction chambers on the first reference plane. The microwell array chip efficiently utilizes the area of the microwell array substrate, so that a volume of a single reaction chamber is increased, furthermore, sensitivity of the microwell array chip can be improved, and detection limit of the microwell array chip can be reduced.

At least one embodiment of the present disclosure provides a microwell array chip, which includes: a microwell array substrate, comprising a first main surface and a second main surface that are oppositely arranged; n reaction chambers, wherein the n reaction chambers are arranged in an array in the microwell array substrate, and are configured to accommodate a sample to be tested, and a shape of an orthographic projection of each of the reaction chambers on a first reference plane where the first main surface is located is a regular N-polygon; and an idle region, arranged around the n reaction chambers; an area of the idle region is divided into n′ virtual units, a shape of an orthographic projection of each of the virtual units on the first reference plane is the same as the shape of the orthographic projection of each of the reaction chambers on the first reference plane, a total volume V′ of the n reaction chambers satisfies the following formula:

$$- 20 {nln ( 1 - α )}^{\frac{1}{n}} \leq V \leq tan \frac{180 °}{N} \times {N ( \sqrt{\frac{{S}_{chip}}{N tan \frac{180 °}{N} ( n + {n}^{′} )}} - X )}^{2} \times n \times h ,$$

In which, (1−α) is a confidence level, S_{chip} is an area of the microwell array substrate, h is a depth of each of the reaction chambers in a direction perpendicular to the first reference plane. N is a positive integer greater than or equal to 3, and X is ½ of a size of an interval between adjacent reaction chambers along a connection line between centers of the adjacent reaction chambers.

For example, the microwell array chip provided by an embodiment of the present disclosure further includes: a support region, configured to set a support structure, the total volume V of the n reaction chambers satisfies the following formula:

$$- 20 {nln ( 1 - α )}^{\frac{1}{n}} \leq V \leq tan \frac{180 °}{N} \times {N ( \sqrt{\frac{{S}_{chip} - {S}_{support}}{N tan \frac{180 °}{N} ( n + {n}^{′} )}} - X )}^{2} \times n \times h ,$$

In which, Sₛᵤₚₚₒᵣₜ is an area of the support region.

For example, the microwell array chip provided by an embodiment of the present disclosure further includes: a reaction region, arranged on a periphery of the support region, the n reaction chambers are located in the reaction region.

For example, the microwell array chip provided by an embodiment of the present disclosure, in the reaction region, distances between centers of orthographic projections of two adjacent reaction chambers on the first reference plane are equal.

For example, in the microwell array chip provided by an embodiment of the present disclosure, a shape of the orthographic projection of the reaction chamber on the first reference plane where the first main surface is located is a regular hexagon, the total volume V of the n reaction chambers satisfies the following formula:

$$- 20 {nln ( 1 - α )}^{\frac{1}{n}} \leq V \leq tan 30 ° \times 6 {( \sqrt{\frac{{S}_{chip}}{N tan 30 ° ( n + {n}^{′} )}} - X )}^{2} \times n \times h ,$$

In which, a value range of X is from 10 microns to 20 microns, and a value range of h is from 190 microns to 320 microns.

For example, in the microwell array chip provided by an embodiment of the present disclosure, a value range of n is from 8000 to 100000.

For example, in the microwell array chip provided by an embodiment of the present disclosure, the total volume V of the n reaction chambers satisfies the following formula:

$$15 μL \leq V \leq tan \frac{180 °}{N} \times {N ( \sqrt{\frac{{S}_{chip}}{N tan \frac{180 °}{N} ( n + {n}^{′} )}} - X )}^{2} \times n \times h .$$

For example, the microwell array chip provided by an embodiment of the present disclosure further includes: a first hydrophobic layer, arranged on the first main surface, an orthographic projection of the first hydrophobic layer on the first reference plane is spaced apart from the orthographic projections of the reaction chambers on the first reference plane.

For example, the microwell array chip provided by an embodiment of the present disclosure further includes: a second hydrophobic layer, arranged on the second main surface, the second hydrophobic layer extends to edges of the reaction chambers.

For example, in the microwell array chip provided by an embodiment of the present disclosure, an orthographic projection of the second hydrophobic layer on a second reference plane where the second main surface is located comprises an opening, and an edge of the second opening is coincided with an edge of one reaction chamber on the second reference plane.

For example, the microwell array chip provided by an embodiment of the present disclosure further includes: a second hydrophobic laver, arranged on the second main surface, the reaction chambers penetrate through the microwell array substrate in a direction perpendicular to the first reference plane, the second hydrophobic layer spans across the reaction chambers, and orthographic projections of the reaction chambers on a second reference plane where the second main surface is located fall within an orthographic projection of the second hydrophobic layer on the second reference plane.

For example, in the microwell array chip provided by an embodiment of the present disclosure, each of the reaction chambers is recessed into the microwell array substrate from the first main surface, and has a chamber bottom located in the microwell array substrate, and a distance between the chamber bottom and the first reference plane is smaller than a thickness of the microwell array substrate.

For example, in the microwell array chip provided by an embodiment of the present disclosure, a contact angle between the first hydrophobic layer and the sample to be tested is smaller than a critical angle of the reaction chamber, the critical angle is an angle between an extension line of a side wall of the reaction chamber and a tangent line of a surface of the sample to be tested which is in contact with the side wall of the reaction chamber.

For example, in the microwell array chip provided by an embodiment of the present disclosure, the chamber bottom comprises at least one exhaust hole, each of the at least one exhaust hole penetrates through the chamber bottom in a direction perpendicular to the first reference plane.

For example, in the microwell array chip provided by an embodiment of the present disclosure, the chamber bottom comprises one exhaust hole, an orthographic projection of the exhaust hole on the second reference plane where the second main surface is located is located at a center of an orthographic projection of the chamber bottom on the second reference plane.

For example, in the microwell array chip provided by an embodiment of the present disclosure, the chamber bottom comprises a plurality of exhaust holes, orthographic projections of the plurality of exhaust holes on the second reference plane where the second main surface is located are arranged around a center of the orthographic projection of the chamber bottom on the second reference plane.

For example, the microwell array chip provided by an embodiment of the present disclosure further includes: a dialysis membrane; and a second hydrophobic layer, the reaction chambers penetrate through the microwell array substrate in a direction perpendicular to the first reference plane, the second hydrophobic layer extends to edges of the reaction chambers, and the dialysis membrane spans across one reaction chamber.

For example, in the microwell array chip provided by an embodiment of the present disclosure, an orthographic projection of the reaction chamber on the second reference plane where the second main surface is located falls within an orthographic projection of the dialysis membrane on the second reference plane.

For example, in the microwell array chip provided by an embodiment of the present disclosure, the dialysis membrane is a flexible dialysis membrane.

For example, in the microwell array chip provided by an embodiment of the present disclosure, the dialysis membrane is located on a side of the second hydrophobic layer close to the second main surface.

For example, in the microwell array chip provided by an embodiment of the present disclosure, the dialysis membrane is located on a side of the second hydrophobic layer away from the second main surface.

For example, in the microwell array chip provided by an embodiment of the present disclosure, an included angle between an inner surface of each of the reaction chambers and the first main surface is greater than 90 degrees.

For example, in the microwell array chip provided by an embodiment of the present disclosure, an inner side surface of each of the reaction chambers comprises a first sub-surface and a second sub-surface in a direction perpendicular to the first reference plane, the second sub-surface is located on a side of the first sub-surface away from the first main surface, an angle between the first sub-surface and the first main surface is greater than 90 degrees, and an included angle between the second sub-surface and the second main surface is greater than 90 degrees.

For example, in the microwell array chip provided by an embodiment of the present disclosure, an inner side surface of each of the reaction chambers comprises a first sub-surface, a second sub-surface, and a third sub-surface in a direction perpendicular to the first reference plane, and the second sub-surface is located on a side of the first sub-surface away from the first main surface, and the third sub-surface is located on a side of the second sub-surface away from the first sub-surface, and an included angle between the first sub-surface and the first main surface is greater than 90 degrees, a plane where the second sub-surface is located is perpendicular to the first reference plane, and an included angle between the third sub-surface and the second main surface is greater than 90 degrees.

For example, in the microwell array chip provided by an embodiment of the present disclosure, an inner side surface of each of the reaction chambers comprises a first sub-surface, a second sub-surface, and a third sub-surface in a direction perpendicular to the first reference plane, the second sub-surface is located on a side of the first sub-surface away from the first main surface, and the third sub-surface is located on a side of the second sub-surface away from the first sub-surface, and an included angle between the first sub-surface and the first main surface is greater than 90 degrees, the second sub-surface is an arc surface, and is recessed toward the microwell array substrate, and an included angle between the third sub-surface and the second main surface is greater than 90 degrees.

For example, in the microwell array chip provided by an embodiment of the present disclosure, an inner surface of each of the reaction chambers is provided with a first hydrophilic membrane and a second hydrophilic membrane, the first hydrophilic membrane and the second hydrophilic membrane are adjacently arranged in a direction perpendicular to the first reference plane, a surface of the first hydrophilic film away from the inner surface of each of the reaction chambers is an arc surface protruding toward a central axis of the reaction chamber, and a surface of the second hydrophilic film away from the inner surface of each of the reaction chambers is an arc surface protruding toward a central axis of each of the reaction chambers.

For example, in the microwell array chip provided by an embodiment of the present disclosure, the inner surface of the each of the reaction chambers is a plane, a thickness of the first hydrophilic film is different in the direction perpendicular to the inner surface, so that the surface of the first hydrophilic membrane away from the inner surface of the reaction chamber is an arc surface, a thickness of the second hydrophilic film is different in the direction perpendicular to the inner surface, so that the surface of the second hydrophilic membrane away from the inner surface of the reaction chamber is an arc surface.

For example, in the microwell array chip provided by an embodiment of the present disclosure, the inner side surface of the reaction chamber comprises a first sub-surface and a second sub-surface in a direction perpendicular to the first reference plane, and the second sub-surface is located on a side of the first sub-surface away from the first main surface, and the first sub-surface is protruded toward the central axis of the reaction chamber, so that the surface of the first hydrophilic membrane away from the inner surface of the reaction chamber is an arc surface, the second sub-surface is protruded toward the central axis of the reaction chamber, so that the surface of the second hydrophilic membrane away from the inner surface of the reaction chamber is an arc surface.

For example, in the microwell array chip provided by an embodiment of the present disclosure, a shape of an orthographic projection of each of the reaction chambers on the first reference plane comprises one selected from the group consisting of a circle, a regular hexagon, and a regular octagon.

For example, in the microwell array chip provided by an embodiment of the present disclosure, the shape of the orthographic projection of the reaction chamber on the first reference plane is a triangle.

For example, the microwell array chip provided by an embodiment of the present disclosure further includes: a first encapsulation film, located on a side of the first main surface away from the second main surface; a second encapsulation film, located on a side of the second main surface away from the first main surface, the first encapsulation film and the second encapsulation film are attached to the microwell array substrate by static electricity or colloid.

For example, the microwell array chip provided by an embodiment of the present disclosure further includes: a photocurable oil, located at an opening of the reaction chamber close to the first main surface, the photocurable oil includes a convex structure, the convex structure is arranged in contact with the first main surface, and is located on a side of the first hydrophobic layer close to the central axis of the reaction chamber.

For example, in the microwell array chip provided by an embodiment of the present disclosure, the microwell array substrate is a flexible substrate.

For example, in the microwell array chip provided by an embodiment of the present disclosure, the microwell array substrate further comprises: a liquid-inlet channel, the n reaction chambers are in communication with the liquid-inlet channel, and a one-way membrane is arranged between each of the reaction chambers and the liquid-inlet channel.

For example, in the microwell array chip provided by an embodiment of the present disclosure, the liquid-inlet channel includes: a liquid-inlet main channel; and n liquid-inlet branch channels, respectively connected with the liquid-inlet main channel, the n liquid-inlet branch channels and the n reaction chambers are arranged in one-to-one correspondence.

For example, in the microwell array chip provided by an embodiment of the present disclosure, the microwell array substrate further comprises: a liquid-inlet channel, comprising a plurality of sub-liquid inlet channels communicated with each other, each of the plurality of sub-liquid inlet channels is communicated with multiple reaction chambers, and a height of the sub-liquid inlet channel is greater than the heights of the corresponding multiple reaction chambers.

For example, in the microwell array chip provided by an embodiment of the present disclosure, the heights of the plurality of sub-liquid inlet channels decrease in sequence.

For example, in the microwell array chip provided by an embodiment of the present disclosure, the heights of the plurality of liquid-inlet channels decrease in sequence from the middle to both sides.

For example, the microwell array chip provided by an embodiment of the present disclosure further includes: a first substrate, located on a side of the microwell array substrate, and spaced apart from the first main surface; and a second substrate, located on a side of the microwell array substrate away from the first substrate, the second substrate comprises a heating electrode, an orthographic projection of the heating electrode on the first reference plane is overlapped with orthographic projections of at least part of the n reaction chambers on the first reference plane.

For example, in the microwell array chip provided by an embodiment of the present disclosure, the first substrate comprises: a first base substrate; and a third hydrophobic layer, located on a side of the first base substrate close to the second substrate.

For example, in the microwell array chip provided by an embodiment of the present disclosure, the second substrate further comprises: a second base substrate; a control electrode, located on the second base substrate; a first insulating layer, located on a side of the control electrode away from the second base substrate; and a second insulating layer, the first insulating layer includes a connection hole, the connection hole exposes at least a part of the control electrode, the heating electrode is located on a side of the first insulating layer away from the second base substrate, and is connected with the control electrode through the connection hole, the second insulating layer is located on a side of the heating electrode away from the first insulating layer, and the microwell array substrate is located on the second insulating layer.

For example, the microwell array chip provided by an embodiment of the present disclosure further includes: a photosensitive sensor, located on a side of the second substrate away from the first substrate, the photosensitive sensor is configured to detect light emitted by the reaction chambers in the microwell array substrate.

At least one embodiment of the present disclosure further provides a detection device, which includes any one of the abovementioned microwell array chip.

For example, the detection device provided by an embodiment of the present disclosure further includes: a first shell, located on a side of the microwell array chip, and spaced apart from the microwell array chip; and a second shell, located on a side of the microwell array chip away from the first shell, and spaced apart from the microwell array chip, a distance between the microwell array chip and the second shell is greater than or equal to the thickness of the microwell array chip.

For example, in the detection device provided by an embodiment of the present disclosure, the second shell comprises a support structure comprising a first platform part and a second platform part, a height of the second platform part is greater than a height of the first platform part, the first platform part is configured to be in contact with a bottom surface of the microwell array chip, and the second platform part is configured to be in contact with a side surface of the microwell array chip.

For example, in the detection device provided by an embodiment of the present disclosure, a shape of an orthographic projection of the first platform part on the first main surface comprises an arc triangle, a shape of an orthographic projection of the second platform part on the first main surface comprises a semicircle, a bottom of the arc triangle connected with the semicircle is a straight line, and the other two sides of the arc triangle are arcs.

For example, in the detection device provided by an embodiment of the present disclosure, the second shell further comprises a positioning circular platform, and the positioning circular platform is configured to be in contact with a side surface of the microwell array chip.

At least one embodiment of the present disclosure further provides a using method of a microwell array chip, wherein the microwell array chip comprises a microwell array substrate, the using method of a microwell array chip includes: introducing a sample to be tested into the microwell array substrate; encapsulating the sample to be tested in the microwell array substrate, the microwell array substrate includes n reaction chambers and a virtual idle region, the n reaction chamber are arranged in an array in the microwell array substrate, and is configured to accommodate the samples to be tested; a shape of an orthographic projection of each of the reaction chambers on the first reference plane where the first main surface is located is a regular N-polygon, the virtual idle region is arranged around the n reaction chambers; an area of the virtual idle region is divided into n′ virtual units, a shape of an orthographic projection of each of the virtual units on the first reference plane is the same as the shape of the orthographic projection of each of the n reaction chambers on the first reference plane, a total volume V of the n reaction chambers satisfies the following formula:

$$- 20 {nln ( 1 - α )}^{\frac{1}{n}} \leq V \leq tan \frac{180 °}{N} \times {N ( \sqrt{\frac{{S}_{chip}}{N tan \frac{180 °}{N} ( n + {n}^{′} )}} - X )}^{2} \times n \times h ,$$

In which, (1−α) is the confidence level. S_{chip} is an area of the microwell array substrate, h is the depth of the reaction chamber in a direction perpendicular to the first reference plane, N is a positive integer greater than or equal to 3, and X is ½ of a size of the interval between adjacent reaction chambers at a connection line between the centers of the adjacent reaction chambers.

For example, in the using method of the microwell array chip provided by an embodiment of the present disclosure, encapsulating the sample to be tested in the microwell array substrate comprises: after introducing the sample to be tested into the microwell array substrate, attaching the first encapsulation film to a side of the first main surface away from the second main surface by static electricity or colloid, and attaching the second encapsulation film on a side of the second main surface away from the first main surface.

For example, in the using method of the microwell array chip provided by an embodiment of the present disclosure, encapsulating the sample to be tested in the microwell array substrate comprises: after introducing the sample to be tested into the microwell array substrate, coating photocurable oil at the opening position of the reaction chamber close to the first main surface; and using ultraviolet light to cure the photocurable oil.

For example, in the using method of the microwell array chip provided by an embodiment of the present disclosure, the microwell array substrate comprises a first sub-flexible microwell array substrate and a second sub-flexible microwell array substrate, a plurality of sample flow channels are comprised between the first sub-flexible microwell array substrate and the second sub-flexible microwell array substrate, introducing the sample to be tested in the microwell array substrate comprises: introducing the sample to be tested through the plurality of sample flow channels.

For example, the using method of the microwell array chip provided by an embodiment of the present disclosure further includes: introducing the sample to be tested into the plurality of sample flow channels by means of vacuuming.

For example, in the using method of the microwell array chip provided by an embodiment of the present disclosure, encapsulating the sample to be tested in the microwell array substrate comprises: using rollers to separate each of the sample flow channels to form a plurality of reaction chambers, and sealing the plurality of reaction chambers.

### BRIEF DESCRIPTION OF DRAWINGS

In order to clearly illustrate the technical solution of the embodiments of the present disclosure, the drawings of the embodiments will be briefly described. It is obvious that the described drawings in the following are only related to some embodiments of the present disclosure and thus are not construed as any limitation to the present disclosure.

FIG. 1 is a planar schematic diagram of a microwell array chip provided by an embodiment of the present disclosure;

FIG. 2 is a cross-sectional schematic diagram of a microwell array chip provided by an embodiment of the present disclosure;

FIG. 3A is a planar schematic diagram of a reaction chamber in another microwell array chip provided by an embodiment of the present disclosure;

FIG. 3B is a cross-sectional schematic diagram of a reaction chamber in another microwell array chip provided by an embodiment of the present disclosure;

FIG. 4A is a cross-sectional schematic diagram of another microwell array chip provided by an embodiment of the present disclosure;

FIG. 4B is a cross-sectional schematic diagram of still another microwell array chip provided by an embodiment of the present disclosure;

FIG. 5 is a cross-sectional schematic diagram of still another microwell array chip provided by an embodiment of the present disclosure;

FIG. 6A is a planar schematic diagram of still another microwell array chip provided by an embodiment of the present disclosure;

FIG. 6B is a cross-sectional schematic diagram of still another microwell array chip provided by an embodiment of the present disclosure;

FIG. 7A is a planar schematic diagram of still another microwell array chip provided by an embodiment of the present disclosure;

FIG. 7B is a cross-sectional schematic diagram of still another microwell array chip provided by an embodiment of the present disclosure;

FIG. 8A is a planar schematic diagram of still another microwell array chip provided by an embodiment of the present disclosure;

FIG. 8B is a cross-sectional schematic diagram of still another microwell array chip provided by an embodiment of the present disclosure;

FIG. 9A is a planar schematic diagram of still another microwell array chip provided by an embodiment of the present disclosure;

FIG. 9B is a cross-sectional schematic diagram of still another microwell array chip provided by an embodiment of the present disclosure;

FIG. 10A is a planar schematic diagram of still another microwell array chip provided by an embodiment of the present disclosure;

FIG. 10B is a cross-sectional schematic diagram of still another microwell array chip provided by an embodiment of the present disclosure;

FIG. 11A is a cross-sectional schematic diagram of still another microwell array chip provided by an embodiment of the present disclosure;

FIG. 11B is a stereoscopic schematic diagram of a reaction chamber in a microwell array chip provided by an embodiment of the present disclosure;

FIG. 11C is a stereoscopic schematic diagram of a reaction chamber in another microwell array chip provided by an embodiment of the present disclosure;

FIG. 12A is a cross-sectional schematic diagram of another microwell array chip provided by an embodiment of the present disclosure;

FIG. 12B is a stereoscopic schematic diagram of a reaction chamber in a microwell array chip provided by an embodiment of the present disclosure;

FIG. 12C is a stereoscopic schematic diagram of a reaction chamber in another microwell array chip provided by an embodiment of the present disclosure;

FIG. 13A is a cross-sectional schematic diagram of another microwell array chip provided by an embodiment of the present disclosure;

FIG. 13B is a stereoscopic schematic diagram of a reaction chamber in a microwell array chip according to an embodiment of the present disclosure;

FIG. 13C is a stereoscopic schematic diagram of a reaction chamber in another microwell array chip provided by an embodiment of the present disclosure;

FIG. 14A is a cross-sectional schematic diagram of another microwell array chip provided by an embodiment of the present disclosure;

FIG. 14B is a stereoscopic schematic diagram of a reaction chamber in a microwell array chip provided by an embodiment of the present disclosure;

FIG. 14C is a stereoscopic schematic diagram of a reaction chamber in another microwell array chip provided by an embodiment of the present disclosure;

FIG. 15 is a cross-sectional schematic diagram of another microwell array chip provided by an embodiment of the present disclosure;

FIG. 16 is a cross-sectional schematic diagram of still another microwell array chip provided by an embodiment of the present disclosure;

FIG. 17 is a structural schematic diagram of a microwell array chip provided by an embodiment of the present disclosure;

FIG. 18 is a cross-sectional schematic diagram of still another microwell array chip provided by an embodiment of the present disclosure;

FIG. 19 is a cross-sectional schematic diagram of still another microwell array chip provided by an embodiment of the present disclosure;

FIG. 20 is a planar schematic diagram of still another microwell array chip provided by an embodiment of the present disclosure;

FIG. 21 is a planar schematic diagram of still another microwell array chip provided by an embodiment of the present disclosure;

FIG. 22 is a planar schematic diagram of still another microwell array chip provided by an embodiment of the present disclosure;

FIG. 23 is a planar schematic diagram of still another microwell array chip provided by an embodiment of the present disclosure;

FIG. 24 is a cross-sectional schematic diagram of still another microwell array chip provided by an embodiment of the present disclosure;

FIG. 25 is a cross-sectional schematic diagram of still another microwell array chip provided by an embodiment of the present disclosure;

FIG. 26 is a cross-sectional schematic diagram of still another microwell array chip provided by an embodiment of the present disclosure;

FIG. 27 is a schematic diagram of a detection device provided by an embodiment of the present disclosure;

FIG. 28 is a schematic diagram of another detection device provided by an embodiment of the present disclosure; and

FIG. 29 is a planar schematic diagram of a second shell in a detection device according to an embodiment of the present disclosure.

### DETAILED DESCRIPTION

In order to make objectives, technical details, and advantages of the embodiments of the present disclosure more clear, the technical solutions of the embodiments will be described in a clearly and fully understandable way in connection with the drawings related to the embodiments of the present disclosure. Apparently, the described embodiments are just a part but not all of the embodiments of the present disclosure. Based on the described embodiments herein, those skilled in the art can obtain other embodiment(s), without any inventive work, which should be within the scope of the present disclosure.

Unless otherwise defined, all the technical and scientific terms used herein have the same meanings as commonly understood by one of ordinary skill in the art to which the present disclosure belongs. The terms “first”, “second”, etc., which are used in the present disclosure, are not intended to indicate any sequence, amount or importance, but distinguish various components. Also, the terms “comprise.” “comprising,” “include,” “including.” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly.

Unless otherwise defined, the features used in the embodiment of the present disclosure, such as “parallel”. “vertical”, and “identical”, all include strictly defined situations such as “parallel”, “vertical”, and “identical”, as well as situations where “roughly parallel”, “roughly vertical”, and “roughly identical” contain certain errors. For example, the above “roughly” can indicate that the difference between the compared objects is 10% of the average value of the compared objects, or within 5%. When the number of a component or an element is not specifically specified in the following embodiment of the present disclosure, it refers to that the component or the element can be one or multiple, or can be understood as at least one. “At least one” refers to one or more, and “multiple” refers to at least two. The “arranged in the same layer” in the embodiment of the present disclosure refers to the relationship between multiple film layers formed by the same material after formed in the same step (such as a one-step patterning process). The term “same layer” here does not always refer to that multiple film layers have the same thickness or have the same height in the cross-sectional view.

In the research, the inventor(s) of the present application has noticed that the main problem of the current digital PCR chip is how to improve the sensitivity, how to reduce the detection limit of the chip; from a chemical perspective, a volume of a single chamber is large enough, the more abundant the sample, the more complete the reaction; on the other hand, if a volume of a single reaction chamber is large, the volume of the chamber per unit area of the chip will be wasted. Therefore, the requirements of the reaction chamber are controlled within a certain range.

In this regard, embodiments of the present disclosure provide a microwell array chip, a using method thereof, and a detection device. The microwell array chip includes a microwell array substrate, the microwell array substrate includes a first main surface and a second main surface that are oppositely arranged, n reaction chambers and an idle region; the n reaction chambers are arranged in an array in the microwell array substrate, and the idle region is arranged around the n reaction chambers; the n reaction chambers are configured to accommodate samples to be tested, and the idle region is divided into n′ virtual units, the shapes of the orthographic projections of the reaction chambers on the first reference plane where the first main surface is located and shapes of orthographic projections of the virtual units on the first reference plane are both regular N-polygons, and a total volume V of the n reaction chambers satisfies the following formula:

$$- 20 {nln ( 1 - α )}^{\frac{1}{n}} \leq V \leq tan \frac{180 °}{N} \times {N ( \sqrt{\frac{{S}_{chip}}{N tan \frac{180 °}{N} ( n + {n}^{′} )}} - X )}^{2} \times n \times h ,$$

in which, (1−α) is a confidence level. S_{chip} is an area of the microwell array substrate, h is a depth of the reaction chamber in the direction perpendicular to the first reference plane. N is a positive integer greater than or equal to 3, X is ½ of a size of the interval between adjacent reaction chambers along a connection line between centers of the adjacent reaction chambers.

In the microwell array chip provided by the embodiment of the present disclosure, because the total volume V of the n reaction chambers satisfies the above formula, the microwell array chip can efficiently utilize the area of the microwell array substrate, so that the volume of the single reaction chamber is increased on the premise that the number of the reaction chambers is sufficiently large, furthermore, the sensitivity of the microwell array chip can be improved, and the detection limit of the microwell array chip can be reduced.

Hereinafter, the microwell array chip, the using method thereof and the detection device provided by the embodiments of the present disclosure will be described in detail with reference to the accompanying drawings.

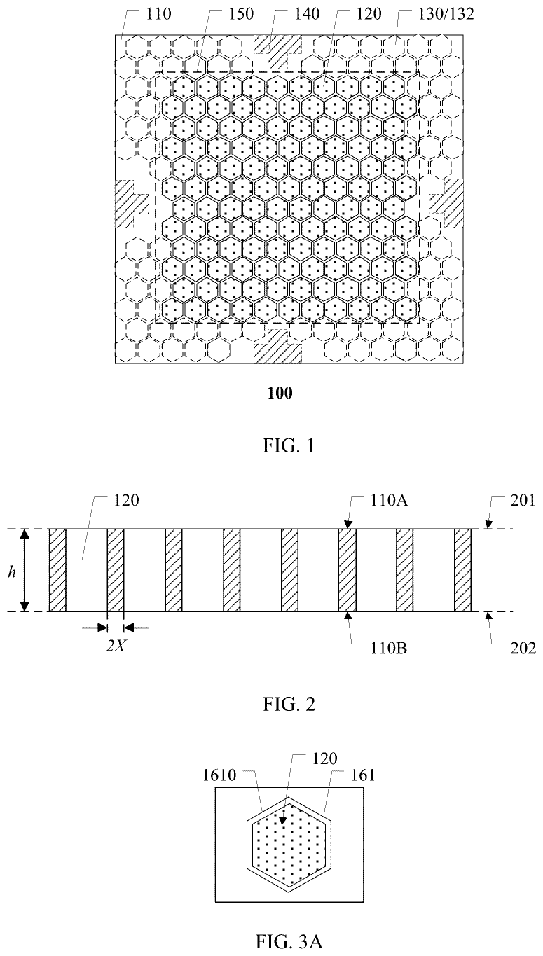

An embodiment of the present disclosure provides a microwell array chip. FIG. 1 is a planar schematic diagram of a microwell array chip provided by an embodiment of the present disclosure. FIG. 2 is a cross-sectional schematic diagram of a microwell array chip provided by an embodiment of the present disclosure.

As illustrated by FIG. 1 and FIG. 2, the microwell array chip 100 includes a microwell array substrate 110; the microwell array substrate 110 includes a first main surface 110A and a second main surface 110B that are oppositely arranged, n reaction chambers 120 and an idle region 130; the n reaction chambers 120 are arranged in an array in the microwell array substrate 110, and the idle region 130 is arranged around the n reaction chambers 120; the n reaction chambers 120 are configured to accommodate the samples to be tested, a shape of an orthographic projections of each of the reaction chambers 120 on a first reference plane 201 where the first main surface 110A is located is a regular N-polygons, an area of the idle region 130 is divided into n′ virtual units 132, a shape of an orthographic projection of each of the virtual units 132 on the first reference plane 201 is the same as the shape of the orthographic projection of each of the reaction chambers 120 on the first reference plane 201, a total volume V of the n reaction chambers 120 satisfies the following formula:

$$- 20 n {ln ( 1 - α )}^{\frac{1}{n}} \leq V \leq tan \frac{180 °}{N} \times {N ( \sqrt{\frac{{S}_{chip}}{N tan \frac{{180}^{∘}}{N} ( n + {n}^{′} )}} - X )}^{2} \times n \times h ,$$

In which, (1−α) is a confidence level, S_{chip} is an area of the microwell array substrate 110, h is a depth of each of the reaction chambers 120 in a direction perpendicular to the first reference plane 201, N is a positive integer greater than or equal to 3, X is ½ of a size of an interval between adjacent reaction chambers 120 along a connection line between the centers of the adjacent reaction chambers 120. It should be noted that in a case that the value of N is infinite, the regular N-polygon can be a circle; in addition, the size and the quantity of the reaction chambers in the microwell array substrate shown in FIG. 1 are only schematic, and the number of reaction chambers can be arranged according to actual requirements.

In the microwell array chip provided by the embodiment of the present disclosure, because the total volume V of the n reaction chambers satisfies the above formula, the microwell array chip can efficiently utilize the area of the microwell array substrate, so that the volume of the single reaction chamber is increased on the premise that the number of the reaction chambers is sufficiently large, furthermore, the sensitivity of the microwell array chip can be improved, and the detection limit of the microwell array chip can be reduced.

In some examples, the total volume V of the n reaction chambers satisfies the following formula:

$$15 μ L \leq V \leq tan \frac{180 °}{N} \times N {( \sqrt{\frac{{S}_{chip}}{N tan \frac{{180}^{∘}}{N} ( n + {n}^{′} )}} - X )}^{2} \times n \times h .$$

In this way, the microwell array chip can efficiently utilize the area of the microwell array substrate, so that the sensitivity of the microwell array chip can be improved, and the detection limit of the microwell array chip can be reduced.

In some examples, as illustrated by FIG. 1 and FIG. 2, the microwell array chip 110 further includes a support region 140, the support region 140 is configured to set a support structure. In this case, the total volume V of the n reaction chambers 120 satisfies the following formula:

$$- 20 n {ln ( 1 - α )}^{\frac{1}{n}} \leq V \leq tan \frac{180 °}{N} \times {N ( \sqrt{\frac{{S}_{chip} - {S}_{support}}{N tan \frac{{180}^{∘}}{N} ( n + {n}^{′} )}} - X )}^{2} \times n \times h ,$$

In which Sₛᵤₚₚₒᵣₜ is the area of the support region 140.

In the microwell array chip provided in the example, the support region may be arranged in contact with a support structure (such as a support structure of a base), so that there is a certain gap between the microwell array substrate and the base, to facilitate entry of liquid (such as the sample to be tested), thus the microwell array substrate can be oil-sealed. It should be noted that different filling patterns are used in the support region in FIG. 1, but the support region can be made of a same material as other regions, that is, the surface of the support region can be the same as the surface of the surrounding idle region. Of course, the embodiments of the present disclosure include but are not limited thereto. A surface of the support region in contact with the support structure may also be coated with other materials or structures, or may be surface treated, to increase friction or other characteristics, and to better contact with the support structure.

In some examples, as illustrated by FIG. 1 and FIG. 2, the microwell array chip 100 further includes a reaction region 150; the reaction region 150 is arranged at the periphery of the support region 140, the n reaction chambers 120 are arranged in the reaction region 150. In this way, no reaction chamber is arranged in the support region of the microwell array chip, and the reaction region is arranged at the periphery of the support region, so that the area of the microwell array substrate can be fully utilized.

For example, as illustrated by FIG. 1 and FIG. 2, four support regions 140 are located on four edges of the microwell array substrate 110, and are respectively located in the middle of the edges, the reaction region 150 is arranged in the middle region of the microwell array substrate 110, and is located around the support region 140.

It should be noted that, in the microwell array chip provided by the embodiment of the present disclosure, the arrangement of the virtual units is to calculate an area of the idle region and the area occupied by the reaction chambers as units, thus the virtual units and the reaction chambers not only have a same shape, but also have a same mutual arrangement. For example, a distance between a virtual unit and a reaction chamber that are adjacent, a distance between adjacent reaction chambers and a distance between adjacent virtual units may be the same. For another example, in a case that the reaction chambers are arranged according to a certain period to form a reaction chamber group (for example, to form a reaction chamber row or a reaction chamber column), the virtual units can also be arranged according to a certain period to form a virtual group. For another example, in a case that the reaction chambers are arranged regularly or misplaced due to their shapes, the virtual units are also arranged regularly or misplaced, to fill the reaction region and the idle region on the entire microwell array substrate. It should be noted that in a case that the microwell array chip includes a support region, the virtual units only need to avoid the support region, and the arrangement of the virtual units in the idle region is still the same as the arrangement of the reaction chamber.

In some examples, as illustrated by FIG. 1 and FIG. 2, in the reaction region 150, the center distances of orthographic projections of two adjacent reaction chambers 120 on the first reference plane 201 are equal. That is, barriers between adjacent reaction chambers in the reaction region are equal, and the reaction chambers are evenly distributed. In this way, the microwell array chip can ensure that volumes of liquids (such as the sample to be tested) entering all of the reaction chambers are consistent, so that the reaction is more precise.

In some examples, as illustrated by FIG. 1 and FIG. 2, the shape of the orthographic projection of each of the reaction chambers 120 on the first reference plane 201 is a regular hexagon, so that the liquid (for example, the sample to be tested) can enter the reaction chamber more easily while having a larger volume. Of course, embodiments of the present disclosure include but are not limited thereto. The shape of the orthographic projection of each of the reaction chambers 120 on the first reference plane 201 may also be a circle, a regular octagon, a regular pentagon, a square or a triangle and so on.

For example, the shape of the orthographic projection of each of the reaction chambers on the first reference plane may be a triangle. Because an angle of each corner of the triangle is small, a surface tension of the sample to be tested can be destroyed, thus it is more convenient for the sample to be tested to enter the reaction chamber.

In some examples, as illustrated by FIG. 1 and FIG. 2, the shapes of the orthographic projections of the reaction chambers 120 on the first reference plane 201 where the first main surface 110A is located and the shapes of the orthographic projections of the virtual units 132 on the first reference plane 201 are all regular hexagons, the total volume V of then reaction chambers satisfies the following formula:

$$- 20 n {ln ( 1 - α )}^{\frac{1}{n}} \leq V \leq tan 30 ° \times 6 {( \sqrt{\frac{{S}_{chip}}{N tan 30 ° - ( n + {n}^{′} )}} - X )}^{2} \times n \times h ,$$

In which, a value range of X is from 10 microns to 20 microns, and a value range of h is from 190 microns to 320 microns.

In the microwell array chip provided in the example, the shape of the orthographic projection of each of the reaction chambers on the first reference plane is a regular hexagon, and because the total volume I′ of the n reaction chambers satisfies the above formula, the microwell array chip can efficiently utilize the area of the microwell array substrate, so that a volume of a single reaction chamber is increased, furthermore, the sensitivity of the microwell array chip can be improved, and the detection limit of the microwell array chip can be reduced.

In some examples, a distance between adjacent reaction chambers 120 may be in a range of 20 microns to 40 microns along a connection line between centers of adjacent reaction chambers 120, for example, 24 microns, 26 microns, 28 microns, 30 microns, 32 microns, 34 microns or 36 microns.

In some examples, a depth of each of the reaction chambers 120 in a direction perpendicular to the first reference plane 201 may be 200 microns, 220 microns, 240 microns, 260 microns, 280 microns or 300 microns.

In some examples, a number of the reaction chambers 120 on one microwell array substrate 110 may ranges from 8.000 to 100,000. In this way, the microwell array chip has higher detection accuracy.

In some examples, the number of reaction chambers 120 on one microwell array substrate 110 may be 8,000, 10,000, 20,000, 40,000, 60,000, 80,000 or 100,000.

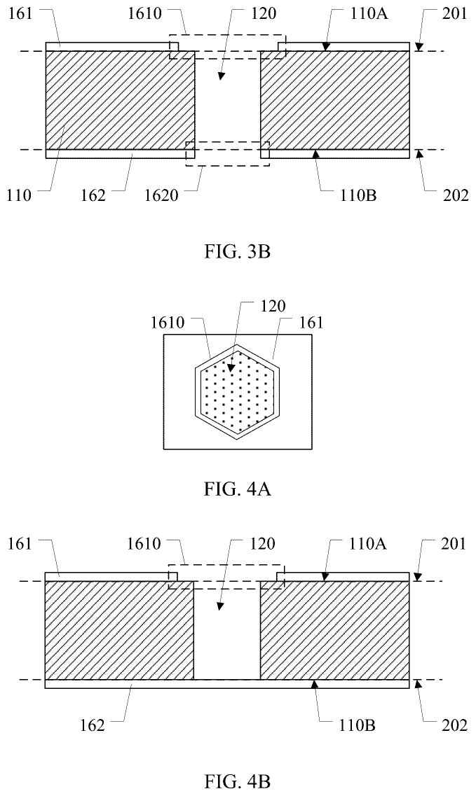

FIG. 3A is a planar schematic diagram of a reaction chamber in another microwell array chip provided by an embodiment of the present disclosure; FIG. 3B is a cross-sectional schematic diagram of a reaction chamber in another microwell array chip provided by an embodiment of the present disclosure.

As illustrated by FIG. 3A and FIG. 3B, the microwell array chip 100 further includes a first hydrophobic layer 161, and the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to edges of the reaction chambers 120, so that it is possible to facilitate the entry of the sample or reagent into the reaction chambers while preventing the sample or reagent from remaining in regions outside the reaction chambers. It should be noted that, the first hydrophobic layer has hydrophobic and lipophilic properties, so that the liquid (such as the sample to be tested) can more easily enter the reaction chambers defined by the microwell array substrate.

For example, a material of the first hydrophobic layer may be resin or silicon nitride, such as epoxy resin. The first hydrophobic layer can also be made of other suitable inorganic or organic materials, as long as the side of the first hydrophobic layer away from the microwell array substrate is hydrophobic.

In some examples, as illustrated by FIG. 3A and FIG. 3B, an orthographic projection of the first hydrophobic layer 161 on the first reference plane 201 where the first main surface 110A is located includes a first opening 1610, an edge of the first opening 1610 is spaced apart from an edge of the reaction chamber 120 on the first reference plane 201, and is located outside the edge of the reaction chamber 120 on the first reference plane 201.

In some examples, as illustrated by FIG. 3A, the shapes of the orthographic projections of the reaction chambers 120 on the first reference plane 201 are regular hexagons, a shape of the first opening 1610 is a regular hexagon, and a side length of the first opening 1610 is greater than a side length of the orthographic projection of each of the reaction chambers 120 on the first reference plane 201.

In some examples, as illustrated by FIG. 3A and FIG. 3B, in the microwell array chip 100, the reaction chambers 120 penetrate through the microwell array substrate 110 in a direction perpendicular to the microwell array substrate 110 (that is, a direction perpendicular to the first reference plane 201). That is, the reaction chambers 120 are through holes penetrating through the microwell array substrate 110.

In some examples, as illustrated by FIG. 3A and FIG. 3B, the microwell array chip 100 further includes a second hydrophobic layer 162; the second hydrophobic layer 162 is located on the second main surface 110B; the second hydrophobic layer 162 extends to edges of the reaction chambers 120. It should be noted that the second hydrophobic layer has hydrophobic and lipophilic properties, in this way, the liquid (such as the sample to be tested) can more easily enter the reaction chambers defined by the microwell array substrate.

For example, a material of the second hydrophobic layer may be resin or silicon nitride, such as epoxy resin. The second hydrophobic layer can also be made of other suitable inorganic or organic materials, as long as a side of the second hydrophobic layer away from the microwell array substrate is hydrophobic.

In some examples, as illustrated by FIG. 3A and FIG. 3B, an orthographic projection of the second hydrophobic layer 162 on the second reference plane 202 where the second main surface 110B is located includes a second opening 1620, and edges of the second opening 1620 are coincided with the edges of the reaction chamber 120 on the second reference plane 202. That is, the second hydrophobic layer is located just between adjacent reaction chambers.

In some examples, as illustrated by FIG. 3A, the shapes of the orthographic projections of the reaction chambers 120 on the first reference plane 201 are regular hexagons, the shape of the second opening 1620 is a regular hexagon, and the side length of the second opening 1620 is equal to the side length of the orthographic projection of each of the reaction chambers 120 on the first reference plane 201.

FIG. 4A is a cross-sectional schematic diagram of another microwell array chip provided by an embodiment of the present disclosure; FIG. 4B is a cross-sectional schematic diagram of still another microwell array chip provided by an embodiment of the present disclosure.

As illustrated by FIG. 4A and FIG. 4B, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 4A and FIG. 4B, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from orthographic projections of the reaction chambers 120 on the first reference plane 201. That is, the first hydrophobic layer 161 does not extend to edges of the reaction chamber 120, so that it is possible to facilitate the entry of the sample or reagent into the reaction chamber while preventing the sample or reagent from remaining in a region outside the reaction chambers. It should be noted that the first hydrophobic layer has hydrophobic and lipophilic properties, in this way, the liquid (such as the sample to be tested) can more easily enter the reaction chamber defined by the microwell array substrate.

For example, the material of the first hydrophobic layer may be resin or silicon nitride, such as epoxy resin. The first hydrophobic layer can also be made of other suitable inorganic or organic materials, as long as a side of the first hydrophobic layer away from the microwell array substrate is hydrophobic.

In some examples, as illustrated by FIG. 4A and FIG. 4B, the orthographic projection of the first hydrophobic layer 161 on the first reference plane 201 where the first main surface 110A is located includes a first opening 1610, an edge of the first opening 1610 is spaced apart from the edge of the reaction chamber 120 on the first reference plane 201, and is located outside the edges of the reaction chamber 120 on the first reference plane 201.

In some examples, as illustrated by FIG. 4A and FIG. 4B, the microwell array chip 100 further includes a second hydrophobic layer 162 located on the second main surface 110B; the second hydrophobic layer 162 spans across the reaction chambers 120, orthographic projections of the reaction chambers 120 on the second reference plane 202 of the second main surface 110B fall within the orthographic projection of the second hydrophobic layer 162 on the second reference plane 202. That is, the microwell array chip can seal the reaction chamber on the side of the second main surface through the second hydrophobic layer, so that the reaction chamber forms a blind hole.

In some examples, as illustrated by FIG. 4A, the shape of the orthographic projection of the reaction chamber 120 on the first reference plane 201 is a regular hexagon; in this case, the shape of the first opening 1610 of the orthographic projection of the first hydrophobic layer 161 on the first reference plane 201 is also a regular hexagon, and the side length of the first opening 1610 is greater than the side length of the orthographic projection of the reaction chamber 120 on the first reference plane 201.

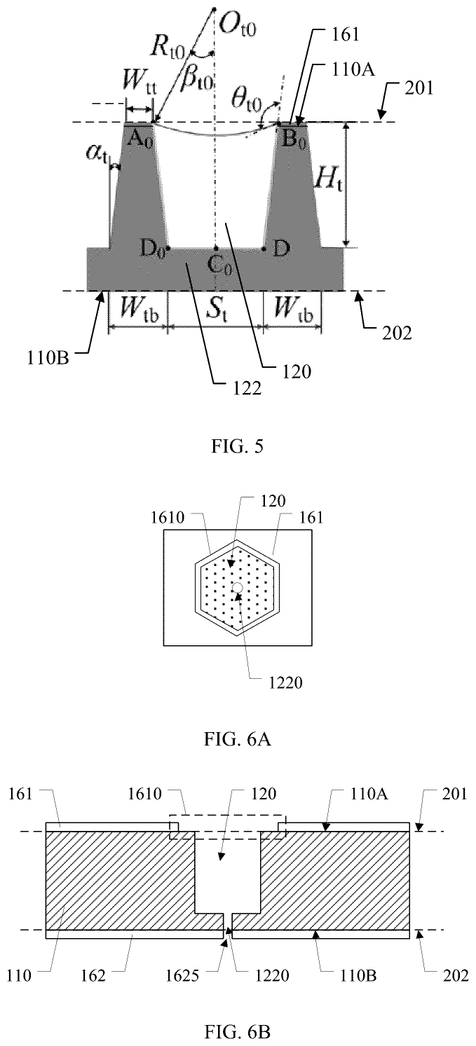

FIG. 5 is a cross-sectional schematic diagram of still another microwell array chip provided by an embodiment of the present disclosure. As illustrated by FIG. 5, the reaction chamber 120 is recessed into the microwell array substrate 110 from the first main surface 110A and has a chamber bottom 122 located in the microwell array substrate 110, a distance between the chamber bottom 122 and the first reference plane 201 is smaller than a thickness of the microwell array substrate 110. That is, the reaction chamber is a blind hole.

In some examples, as illustrated by FIG. 5, the microwell array chip 100 further includes a first hydrophobic layer 161, and the first hydrophobic layer 161 is arranged on the first main surface 110A. A contact angle θₑ between the first hydrophobic layer 161 and the sample to be tested is smaller than a critical angle θₜ₀ of the reaction chamber 120, the critical angle θₜ₀ is an angle between an extension line of the side wall of the reaction chamber 120 and a tangent of the surface of the sample to be tested in contact with the side wall of the reaction chamber 120. In this way, the microwell array chip can make the sample to be tested better enter the reaction chamber.

In some examples, as illustrated by FIG. 5, two adjacent reaction chambers 120 are arranged along the first direction, a dimension of a side wall between two adjacent reaction chambers 120 along the first direction on the first reference plane 201 is Wₜₜ, a dimension of the side wall between two adjacent reaction chambers 120 along the first direction on the second reference plane 202 is W_{tb}, a size of an orthographic projection of a chamber bottom 122 of the reaction chamber 120 on the second reference plane 202 along the first direction is Sₜ, and an angle between the side wall of the reaction chamber 120 and the direction perpendicular to the second reference plane 202 is αₜ; in this case, the critical angle θₜ₀ of the reaction chamber satisfies the following formula:

$${θ}_{t 0} = arctan \frac{{H}_{t}}{{S}_{t} + {W}_{tb} - {W}_{tt}} + \frac{π}{2} + {α}_{t} .$$

FIG. 6A is a planar schematic diagram of still another microwell array chip provided by an embodiment of the present disclosure; and FIG. 6B is a cross-sectional schematic diagram of still another microwell array chip provided by an embodiment of the present disclosure.

As illustrated by FIG. 6A and FIG. 6B, the reaction chamber 120 is recessed into the microwell array substrate 110 from the first main surface 110A and has a chamber bottom 122 located in the microwell array substrate 110, and a distance between the chamber bottom 122 and the first reference plane 201 is smaller than a thickness of the microwell array substrate 110. That is, the reaction chamber is a blind hole.

In some examples, as illustrated by FIG. 6A and FIG. 6B, the chamber bottom 122 includes at least one exhaust hole 1220, each of the at least one exhaust hole 1220 penetrates through the chamber bottom 122 in a direction perpendicular to the first reference plane 201. In this way, in a case that the liquid (such as the sample to be tested) enters the reaction chamber, the exhaust hole can be used for the discharge of gas, so that it is easier for the liquid to enter the reaction chamber, and the speed of the liquid enters the reaction chamber is increased.

In some examples, as illustrated by FIG. 6A and FIG. 6B, the chamber bottom 122 includes one exhaust hole 1220, an orthographic projection of the exhaust hole 1220 on the second reference plane 202 where the second main surface 110B is located is located at the center of the orthographic projection of the chamber bottom 122 on the second reference plane 202. In this way, air can be quickly exhausted from the exhaust hole of the reaction chamber, so that the speed that the liquid (such as the sample to be tested) enters the reaction chamber is increased.

In some examples, as illustrated by FIG. 6A and FIG. 6B, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chambers 120, so that it is possible to facilitate the entry of the sample or reagent into the reaction chambers while preventing the sample or reagent from remaining in the region outside the reaction chambers. It should be noted that the first hydrophobic layer has hydrophobic and lipophilic properties, so that the liquid (such as the sample to be tested) can more easily enter the reaction chambers defined by the microwell array substrate.

For example, a material of the first hydrophobic layer may be resin or silicon nitride, such as epoxy resin. The first hydrophobic layer can also be made of other suitable inorganic or organic materials, as long as a side of the first hydrophobic layer away from the microwell array substrate is hydrophobic.

In some examples, as illustrated by FIG. 6A and FIG. 6B, an orthographic projection of the first hydrophobic layer 161 on the first reference plane 201 where the first main surface 110A is located includes a first opening 1610, and edges of the first opening 1610 are spaced apart from the edges of the reaction chamber 120 on the first reference plane 201, and are located outside the edges of the reaction chamber 120 on the first reference plane 201.

In some examples, as illustrated by FIG. 6A and FIG. 6B, the microwell array chip 100 further includes a second hydrophobic layer 162, which is located on the second main surface 110B; the second hydrophobic layer 162 also includes an exhaust opening 1625, and the exhaust opening 1625 is communicated with the aforementioned exhaust hole 1220.

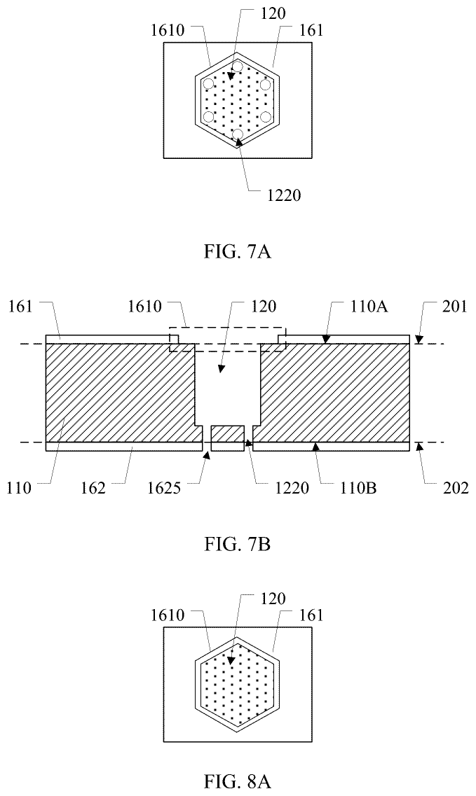

FIG. 7A is a planar schematic diagram of still another microwell array chip provided by an embodiment of the present disclosure; and FIG. 7B is a cross-sectional schematic diagram of still another microwell array chip provided by an embodiment of the present disclosure.

As illustrated by FIG. 7A and FIG. 7B, the reaction chamber 120 is recessed into the microwell array substrate 110 from the first main surface 110A, and has a chamber bottom 122 located in the microwell array substrate 110, and a distance between the chamber bottom 122 and the first reference plane 201 is smaller than a thickness of the microwell array substrate 110. That is, the reaction chamber is a blind hole.

In some examples, as illustrated by FIG. 7A and FIG. 7B, the chamber bottom 122 includes a plurality of exhaust holes 1220, each of the plurality of exhaust holes 1220 penetrates through the chamber bottom 122 in a direction perpendicular to the first reference plane 201; orthographic projections of the plurality of exhaust holes 1220 on the second reference plane 202 where the second main surface 110B is located are arranged around the center of the orthographic projection of the chamber bottom 122 on the second reference plane 202. In this way, in a case that the liquid (such as a sample to be tested) enters the reaction chamber, the plurality of exhaust holes can be used for gas discharge at the same time, so that it is easier for the liquid to enter the reaction chamber, and the speed that the liquid enters the reaction chamber is increased.

In some examples, as illustrated by FIG. 7A, the shape of the orthographic projection of the reaction chamber 120 on the second reference plane 202 is a regular hexagon; in this case, the orthographic projections of the six exhaust holes 1220 on the second reference plane 202 are arranged at six corners of the orthographic projection of the reaction chamber 120 on the second reference plane 202.

In some examples, as illustrated by FIG. 7A and FIG. 7B, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chambers 120, so that it is possible to facilitate the entry of the sample or reagent into the reaction chamber while preventing the sample or reagent from remaining in the area outside the reaction chamber.

In some examples, as illustrated by FIG. 7A and FIG. 7B, an orthographic projection of the first hydrophobic layer 161 on the first reference plane 201 where the first main surface 110A is located includes a first opening 1610, edges of the first opening 1610 are spaced apart from the edges of the reaction chamber 120 on the first reference plane 201, and are located outside the edges of the reaction chamber 120 on the first reference plane 201.

In some examples, as illustrated by FIG. 7A and FIG. 7B, the microwell array chip 100 further includes a second hydrophobic layer 162, which is located on the second main surface 110B; the second hydrophobic layer 162 includes a plurality of exhaust openings 1625, the plurality of exhaust openings 1625 are arranged in one-to-one correspondence with the plurality of exhaust holes 1220, and each of the plurality of exhaust openings 1625 is communicated with a corresponding exhaust hole 1220.

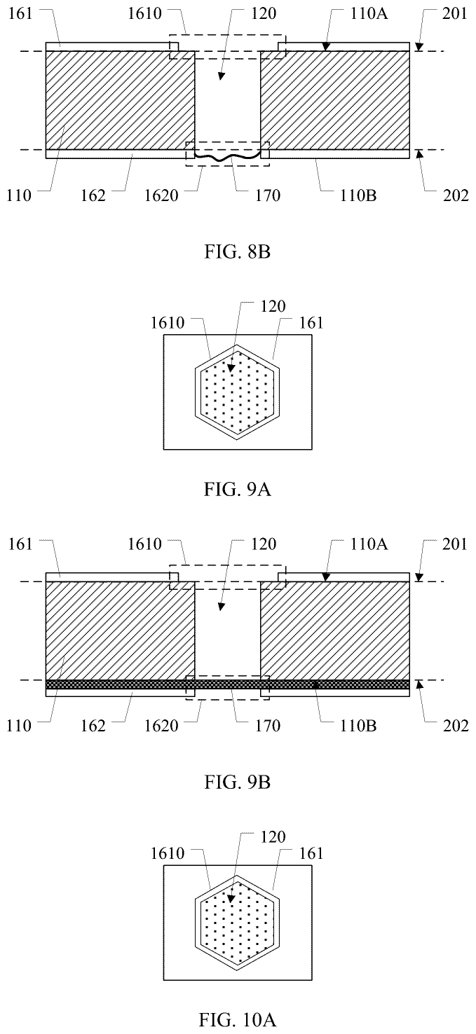

FIG. 8A is a planar schematic diagram of still another microwell array chip provided by an embodiment of the present disclosure; and FIG. 8B is a cross-sectional schematic diagram of still another microwell array chip provided by an embodiment of the present disclosure.

As illustrated by FIG. 8A and FIG. 8B, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 8A and FIG. 8B, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the sample or the reagent entering into the reaction chamber while preventing the sample or the reagent from remaining in the region outside the reaction chamber.

In some examples, as illustrated by FIG. 8A and FIG. 8B, the orthographic projection of the first hydrophobic layer 161 on the first reference plane 201 where the first main surface 110A is located includes the first opening 1610, and the edges of the first opening 1610 are spaced apart from the edges of the reaction chamber 120 on the first reference plane 201, and are located on the outside of the edges of the reaction chamber 120 on the first reference plane 201.

In some examples, as illustrated by FIG. 8A and FIG. 8B, the microwell array chip 100 further includes a dialysis membrane 170 and a second hydrophobic layer 162; the reaction chamber 120 penetrates through the microwell array substrate 110 in the direction perpendicular to the first reference plane 201, the second hydrophobic layer 162 extends to the edges of the reaction chamber 120, and the dialysis membrane 170 spans across the reaction chamber 120. In this way, the dialysis membrane allows the gas in the reaction chamber to be discharged, but the liquid in the reaction chamber is not allowed to flow out, so that the liquid (such as the sample to be tested) can be prevented from flowing out of the reaction chamber while increasing the speed of the liquid entering the reaction chamber.

In some examples, as illustrated by FIG. 8A and FIG. 8B, the orthographic projection of the reaction chamber 120 on the second reference plane 202 where the second main surface 110B is located falls within the orthographic projection of the dialysis membrane 170 on the second reference plane 202.

In some examples, as illustrated by FIG. 8A and FIG. 8B, the dialysis membrane 170 is a flexible dialysis membrane. In a case that the dialysis membrane is a flexible dialysis membrane, the flexible dialysis membrane can better contact with the liquid (such as the sample to be tested), so that the gas in the reaction chamber is exhausted. Of course, the embodiments of the present disclosure include but are not limited thereto, and the dialysis membrane may also be a thin film with certain rigidity.

FIG. 9A is a planar schematic diagram of still another microwell array chip provided by an embodiment of the present disclosure; and FIG. 9B is a cross-sectional schematic diagram of still another microwell array chip provided by an embodiment of the present disclosure.

As illustrated by FIG. 9A and FIG. 9B, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 9A and FIG. 9B, the microwell array chip 100 further includes a first hydrophobic layer 161, the first hydrophobic layer 161 is arranged on the first main surface 110A; and an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the entry of the sample or the reagent into the reaction chamber while preventing the sample or the reagent from remaining in the region outside the reaction chamber.

In some examples, as illustrated by FIG. 9A and FIG. 9B, the orthographic projection of the first hydrophobic layer 161 on the first reference plane 201 where the first main surface 110A is located includes a first opening 1610, edges of the first opening 1610 are spaced apart from the edges of the reaction chamber 120 on the first reference plane 201, and are located outside the edges of the reaction chamber 120 on the first reference plane 201.

In some examples, as illustrated by FIG. 9A and FIG. 9B, the microwell array chip 100 further includes a dialysis membrane 170 and a second hydrophobic layer 162; the reaction chamber 120 penetrates through the microwell array substrate 110 in the direction perpendicular to the first reference plane 201, the second hydrophobic layer 162 extends to the edges of the reaction chamber 120, and the dialysis membrane 170 spans across the reaction chamber 120. The dialysis membrane 170 is located on a side of the second hydrophobic layer 162 close to the second main surface 110B.

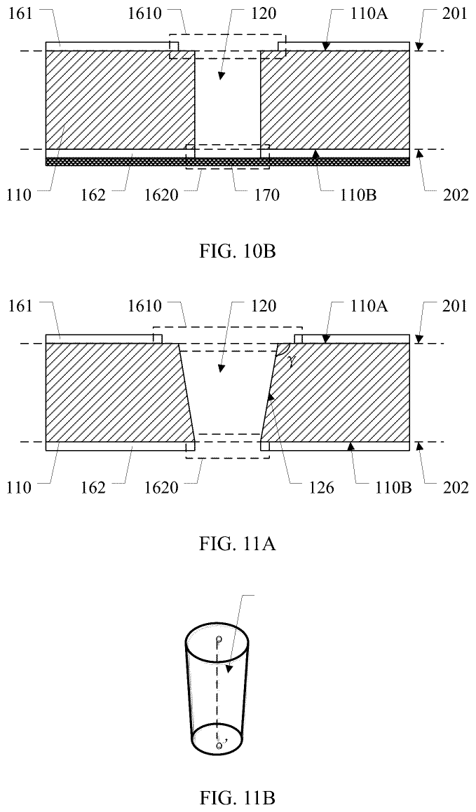

FIG. 10A is a planar schematic diagram of still another microwell array chip provided by an embodiment of the present disclosure; and FIG. 10B is a cross-sectional schematic diagram of still another microwell array chip provided by an embodiment of the present disclosure.

As illustrated by FIG. 10A and FIG. 10B, in the microwell array chip 100, the reaction chamber 120 penetrates through the microwell array substrate 110 in the direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 10A and FIG. 10B, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projection of the reaction chamber 120 on the first reference plane 201. That is, the first hydrophobic layer 161 does not extend to the edges of the reaction chamber 120, so that it is possible to facilitate the sample or the reagent entering into the reaction chamber while preventing the sample or the reagent from remaining in the region outside the reaction chamber.

In some examples, as illustrated by FIG. 10A and FIG. 10B, the orthographic projection of the first hydrophobic layer 161 on the first reference plane 201 where the first main surface 110A is located includes a first opening 1610, edges of the first opening 1610 are spaced apart from the edges of the reaction chamber 120 on the first reference plane 201, and are located outside the edges of the reaction chamber 120 on the first reference plane 201.

In some examples, as illustrated by FIG. 10A and FIG. 10B, the microwell array chip 100 further includes a dialysis membrane 170 and a second hydrophobic layer 162; the reaction chamber 120 penetrates through the microwell array substrate 110 in the direction perpendicular to the first reference plane 201, the second hydrophobic layer 162 extends to the edges of the reaction chamber 120, and the dialysis membrane 170 spans across the reaction chamber 120. The dialysis membrane 170 is located on a side of the second hydrophobic layer 162 away from the second main surface 110B.

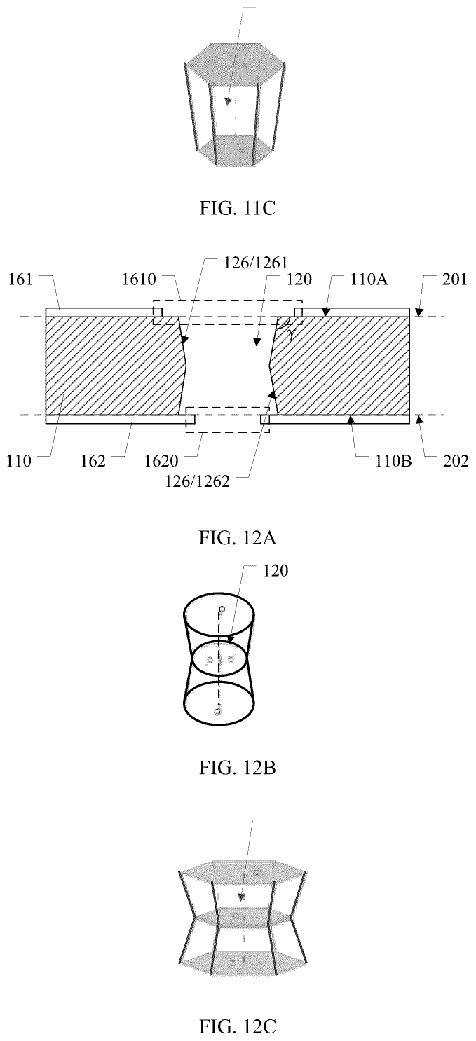

FIG. 11A is a cross-sectional schematic diagram of still another microwell array chip provided by an embodiment of the present disclosure; FIG. 11B is a stereoscopic schematic diagram of a reaction chamber in a microwell array chip provided by an embodiment of the present disclosure; and FIG. 11C is a stereoscopic schematic diagram of a reaction chamber in another microwell array chip provided by an embodiment of the present disclosure.

As illustrated by FIG. 11A, FIG. 11B and FIG. 11C, in the microwell array chip 100, the reaction chamber 120 penetrates through the microwell array substrate 110 in the direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 11A, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the sample or the reagent entering into the reaction chamber while preventing the sample or the reagent from remaining in the region outside the reaction chamber.

In some examples, as illustrated by FIG. 11A, the microwell array chip 100 further includes a second hydrophobic layer 162; the second hydrophobic layer 162 is located on the second main surface 110B; and the second hydrophobic layer 162 extends to the edges of the reaction chamber 120.

In some examples, as illustrated by FIG. 11A, FIG. 11B and FIG. 11C, an angle γ between an inner side surface 126 of the reaction chamber 120 and the first main surface 110A is greater than 90 degrees. That is, a size of the part of the reaction chamber 120 close to the first main surface 110A is relatively large. In this way, the reaction chamber facilitates the entry of the liquid, thereby increasing the speed at which the liquid (such as the sample to be tested) enters the reaction chamber.

In some examples, as illustrated by FIG. 11B, a shape of a cross section of the reaction chamber 120 cut by the first reference plane 201 where the first main surface 110A is located is circular; a shape of a cross section of the reaction chamber 120 cut by the second reference plane 202 where the second main surface 110B is located is circular; moreover, a diameter of the cross section of the reaction chamber 120 cut by the first reference plane 201 is greater than a diameter of the cross section of the reaction chamber 120 cut by the second reference plane 202.

In some examples, as illustrated by FIG. 11C, the shape of the cross section of the reaction chamber 120 cut by the first reference plane 201 where the first main surface 110A is located is a regular hexagon; the shape of the cross section of the reaction chamber 120 cut by the second reference plane 202 where the second main surface 110B is located is a regular hexagon; moreover, a side length of the cross section of the reaction chamber 120 cut by the first reference plane 201 is longer than a side length of the cross section of the reaction chamber 120 cut by the second reference plane 202.

FIG. 12A is a cross-sectional schematic diagram of another microwell array chip provided by an embodiment of the present disclosure; FIG. 12B is a stereoscopic schematic diagram of a reaction chamber in a microwell array chip provided by an embodiment of the present disclosure; and FIG. 12C is a stereoscopic schematic diagram of a reaction chamber in another microwell array chip provided by an embodiment of the present disclosure.

As illustrated by FIG. 12A. FIG. 12B and FIG. 12C, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 12A, the microwell array chip 100 further includes a first hydrophobic layer 161, the first hydrophobic layer 161 is arranged on the first main surface 110A; the orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the entry of the sample or the reagent into the reaction chamber while preventing the sample or the reagent from remaining in the region outside the reaction chambers.

In some examples, as illustrated by FIG. 12A, the microwell array chip 100 further includes a second hydrophobic layer 162; the second hydrophobic layer 162 is located on the second main surface 110B; and the second hydrophobic layer 162 extends to the edges of the reaction chamber 120.

In some examples, as illustrated by FIG. 12A. FIG. 12B and FIG. 12C, an inner side surface 126 of the reaction chamber 120 includes a first sub-surface 1261 and a second sub-surface 1262 in a direction perpendicular to the first reference plane 201, the second sub-surface 1262 is located on a side of the first sub-surface 1261 away from the first main surface 110A, and an angle between the first sub-surface 1261 and the first main surface 110A is greater than 90 degrees, an included angle between the second sub-surface 1262 and the second main surface 110B is greater than 90 degrees. That is, a size of the part of the reaction chamber 120 close to the first main surface 110A is relatively large, a size of the part of the reaction chamber 120 close to the second main surface 110B is relatively large, a size of the part in the middle of the reaction chamber 120 is relatively small. In this way, on the one hand, the reaction chamber facilitates the entry of the liquid, on the other hand, it is conducive to the discharge of gas, so that the speed at which the liquid (such as the sample to be tested) enters the reaction chamber can be further increased.

In some examples, as illustrated by FIG. 12B, a shape of a cross section of the reaction chamber 120 cut by the first reference plane 201 where the first main surface 110A is located is circular; a shape of a cross section of the reaction chamber 120 cut by the second reference plane 202 where the second main surface 110B is located is circular; and a shape of a cross section of the reaction chamber 120 cut by the third reference plane 203 which is parallel to the first reference plane 201 and the second reference plane 202 and located between the first reference plane 201 and the second reference plane 202 is circular; and a diameter of the cross section of the reaction chamber 120 cut by the first reference plane 201 and the diameter of the cross section of the reaction chamber 120 cut by the second reference plane 202 are larger than a diameter of the cross section of the reaction chamber 120 parallel to the first reference plane 201 and the second reference plane 202 and cut by the third reference plane 203 located between the first reference plane 201 and the second reference plane 202.

In some examples, as illustrated by FIG. 12C, the shape of the cross section of the reaction chamber 120 cut by the first reference plane 201 where the first main surface 110A is located is a regular hexagon; the shape of the cross section of the reaction chamber 120 cut by the second reference plane 202 where the second main surface 110B is located is a regular hexagon; and a shape of a cross section of the reaction chamber 120 cut by the third reference plane 203 which is parallel to the first reference plane 201 and the second reference plane 202 and located between the first reference plane 201 and the second reference plane 202 is a regular hexagon; and, a side length of the cross section of the reaction chamber 120 cut by the first reference plane 201 and a side length of the cross section of the reaction chamber 120 cut by the second reference plane 202 are both greater than a side length of the cross section of the reaction chamber 120 cut by the third reference plane 203.

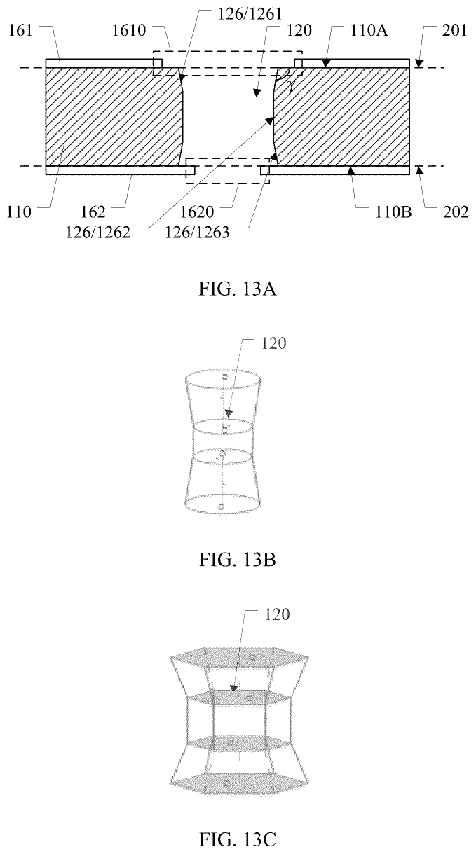

FIG. 13A is a cross-sectional schematic diagram of another microwell array chip provided by an embodiment of the present disclosure; FIG. 13B is a stereoscopic schematic diagram of a reaction chamber in a microwell array chip according to an embodiment of the present disclosure; and FIG. 13C is a stereoscopic schematic diagram of a reaction chamber in another microwell array chip provided by an embodiment of the present disclosure.

As illustrated by FIG. 13A. FIG. 13B and FIG. 13C, in the microwell array chip 100, the reaction chamber 120 penetrates through the microwell array substrate 110 in the direction perpendicular to the microwell array substrate 110. That is, the reaction chamber 120 is a through hole penetrating through the microwell array substrate 110.

In some examples, as illustrated by FIG. 13A, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the entry of the sample or the reagent into the reaction chamber while preventing the sample or the reagent from remaining in the region outside the reaction chamber.

In some examples, as illustrated by FIG. 13A, the microwell array chip 100 further includes a second hydrophobic layer 162; the second hydrophobic layer 162 is located on the second main surface 110B; and the second hydrophobic layer 162 extends to the edges of the reaction chamber 120.

In some examples, as illustrated by FIG. 13A. FIG. 13B, and FIG. 13C, an inner side surface 126 of the reaction chamber 120 includes a first sub-surface 1261, a second sub-surface 1262 and a third sub-surface 1263 in the direction perpendicular to the first reference plane 201, the second sub-surface 1262 is located on a side of the first sub-surface 1261 away from the first main surface 110A, and the third sub-surface 1263 is located on the side of the second sub-surface 1262 away from the first sub-surface 1261. An included angle between the first sub-surface 1261 and the first main surface 110A is greater than 90 degrees, a plane of the second sub-surface 1262 is perpendicular to the first reference plane 201, and an angle between the third sub-surface 1263 and the second main surface 110B is greater than 90 degrees. In this case, the size of the part of the reaction chamber 120 close to the first main surface 110A is relatively large, the size of the part of the reaction chamber 120 close to the second main surface 110B is relatively large, and the size of the part in the middle of the reaction chamber 120 is relatively small. In this way, on the one hand, the reaction chamber facilitates the entry of liquid, on the other hand, the reaction chamber facilitates the discharge of gas, so that the speed that the liquid (such as a sample to be tested) enters the reaction chamber can be further increased. In addition, because the plane of the second sub-surface is perpendicular to the first reference plane, the middle part of the reaction chamber is convenient for storing liquid.

In some examples, as illustrated by FIG. 13B, the shape of the cross section of the reaction chamber 120 cut by the first reference plane 201 where the first main surface 110A is located is circular; the shape of the cross section of the reaction chamber 120 cut by the second reference plane 202 where the second main surface 110B is located is circular; and the shape of the cross section of the reaction chamber 120 cut by the third reference plane 203 which is parallel to the first reference plane 201 and the second reference plane 202 and located between the first reference plane 201 and the second reference plane 202 is circular; and the diameter of the cross section of the reaction chamber 120 cut by the first reference plane 201 and the diameter of the cross section of the reaction chamber 120 cut by the second reference plane 202 are larger than the diameter of the cross section of the reaction chamber 120 cut by the third reference plane 203 which is parallel to the first reference plane 201 and the second reference plane 202 and located between the first reference plane 201 and the second reference plane 202.

In some examples, as illustrated by FIG. 13C, the shape of the cross section of the reaction chamber 120 cut by the first reference plane 201 where the first main surface 110A is located is a regular hexagon; the shape of the cross section of the reaction chamber 120 cut by the second reference plane 202 where the second main surface 110B is located is a regular hexagon; the shape of the reaction chamber 120 cut by the third reference plane 203 which is parallel to the first reference plane 201 and the second reference plane 202, and located between the first reference plane 201 and the second reference plane 202 is a regular hexagon; and a side length of the cross section of the reaction chamber 120 cut by the first reference plane 201 and a side length of the cross section of the reaction chamber 120 cut by the second reference plane 202 are both greater than a side length of the cross section of the reaction chamber 120 cut by the third reference plane 203.

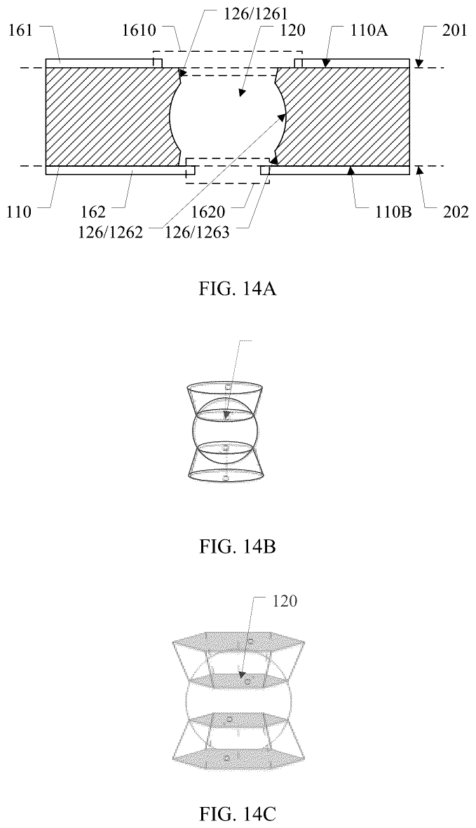

FIG. 14A is a cross-sectional schematic diagram of another microwell array chip provided by an embodiment of the present disclosure; FIG. 14B is a stereoscopic schematic diagram of a reaction chamber in a microwell array chip provided by an embodiment of the present disclosure; and FIG. 14C is a stereoscopic schematic diagram of a reaction chamber in another microwell array chip provided by an embodiment of the present disclosure.

As illustrated by FIG. 14A, FIG. 14B and FIG. 14C, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110.

In some examples, as illustrated by FIG. 14A, the microwell array chip 100 further includes a first hydrophobic layer 161, the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201. That is, the first hydrophobic layer 161 does not extend to the edges of the reaction chamber 120, so that it is possible to facilitate the entry of the sample or the reagent into the reaction chamber while preventing the sample or the reagent from remaining in the region outside the reaction chamber.

In some examples, as illustrated by FIG. 14A, the microwell array chip 100 further includes a second hydrophobic layer 162; the second hydrophobic layer 162 is located on the second main surface 110B; the second hydrophobic layer 162 extends to the edges of the reaction chamber 120.

In some examples, as illustrated by FIG. 14A. FIG. 14B, and FIG. 14C, the inner side surface 126 of the reaction chamber 120 includes a first sub-surface 1261, a second sub-surface 1262 and a third sub-surface 1263 in a direction perpendicular to the first reference plane 201, the second sub-surface 1262 is located on a side of the first sub-surface 1261 away from the first main surface 110A, and the third sub-surface 1263 is located on a side of the second sub-surface 1262 away from the first sub-surface 1261. An included angle between the first sub-surface 1261 and the first main surface 110A is greater than 90 degrees, the second sub-surface 1262 is an arc surface, and is concave toward the microwell array substrate 110, an included angle between the third sub-surface 1263 and the second main surface 110B is greater than 90 degrees. In this case, a size of the part of the reaction chamber 120 close to the first main surface 110A is relatively large, a size of the part of the reaction chamber 120 close to the second main surface 110B is relatively large, a size of the part in the middle of the reaction chamber 120 is relatively small. In this way, on the one hand, the reaction chamber is conducive to the entry of liquid, and on the other hand, the reaction chamber is conducive to the discharge of gas, so that the speed that the liquid (such as the sample to be tested) enters the reaction chamber can be further increased. In addition, because the second sub-surface is an arc surface and is concave toward the microwell array substrate, the middle part of the reaction chamber is convenient for storing liquid.

In some examples, as illustrated by FIG. 14B, the shape of the cross section of the reaction chamber 120 cut by the first reference plane 201 where the first main surface 110A is located is circular; the shape of the cross section of the reaction chamber 120 cut by the second reference plane 202 where the second main surface 110B is located is circular; and the shape of the cross section of the reaction chamber 120 cut by the third reference plane 203 which is parallel to the first reference plane 201 and the second reference plane 202 and located between the first reference plane 201 and the second reference plane 202 is circular; and the diameter of the cross section of the reaction chamber 120 cut by the first reference plane 201 and the diameter of the cross section of the reaction chamber 120 cut by the second reference plane 202 are larger than the diameter of the cross section of the reaction chamber 120 of the reaction chamber 120 cut by the third reference plane 203 which is parallel to the first reference plane 201 and the second reference plane 202 and located between the first reference plane 201 and the second reference plane 202.

In some examples, as illustrated by FIG. 14C, the shape of the cross section of the reaction chamber 120 cut by the first reference plane 201 where the first main surface 110A is located is a regular hexagon; the shape of the cross section of the reaction chamber 120 cut by the second reference plane 202 where the second main surface 110B is located is a regular hexagon; and the shape of the cross section of the reaction chamber 120 cut by the third reference plane 203 which is parallel to the first reference plane 201 and the second reference plane 202 and located between the first reference plane 201 and the second reference plane 202 is a regular hexagon; and the side length of the cross section of the reaction chamber 120 cut by the first reference plane 201 and the side length of the cross section of the reaction chamber 120 cut by the second reference plane 202 are both greater than the side length of the cross section of the reaction chamber 120 cut by the third reference plane 203.

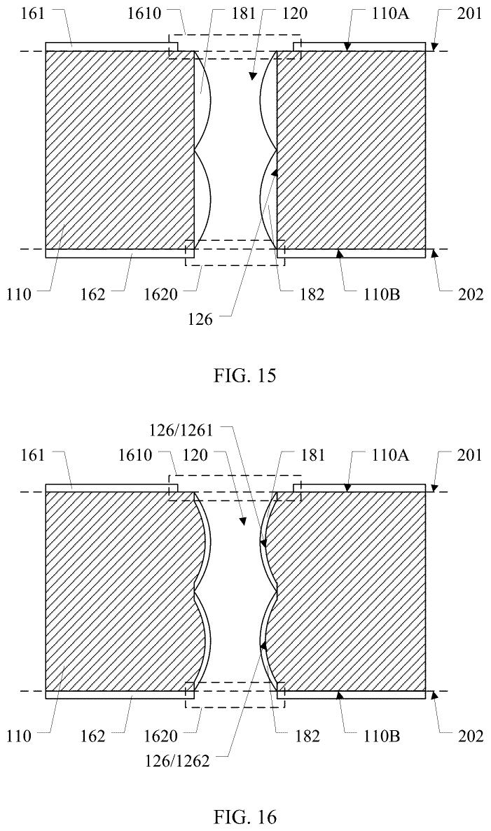

FIG. 15 is a cross-sectional schematic diagram of another microwell array chip provided by an embodiment of the present disclosure. As illustrated by FIG. 15, in the microwell array chip 100, the reaction chamber 120 penetrates through the microwell array substrate 110 in a direction (that is, the direction perpendicular to the first reference plane 201) perpendicular to the microwell array substrate 110. That is, the reaction chamber 120 is a through hole penetrating through the microwell array substrate 110. The inner surface 126 of the reaction chamber 120 is provided with a first hydrophilic film 181 and a second hydrophilic film 182, the first hydrophilic film 181 and the second hydrophilic film 182 are adjacently arranged in the direction perpendicular to the first reference plane 201, a surface of the first hydrophilic film 181 away from the inner surface 126 of the reaction chamber 120 is an arc surface protruding toward a central axis of the reaction chamber 120, a surface of the second hydrophilic membrane 182 away from the inner surface 126 of the reaction chamber 120 is an arc surface protruding toward the central axis of the reaction chamber 120. In this way, on the one hand, the first hydrophilic membrane and the second hydrophilic membrane can facilitate that the liquid (such as the sample to be tested) enters into the reaction chamber and are convenient for storing the liquid; on the other hand, a surface of the first hydrophilic membrane away from the inner surface of the reaction chamber is an arc surface protruding toward the central axis of the reaction chamber, so that a size of a part of the reaction chamber close to the first main surface is larger, which is more conducive for the liquid to enter the reaction chamber. Similarly, a surface of the second hydrophilic membrane away from the inner surface of the reaction chamber is arc-shaped, so that a size of a part of the reaction chamber close to the second main surface is larger, which is conducive to gas discharge; at the same time, a recessed portion (that is, the adjacent part of the first hydrophilic film and the second hydrophilic film) recessed toward the microwell array substrate can also be formed between the first hydrophilic film and the second hydrophilic film, so that it is more convenient for storing the liquid.

In some examples, as illustrated by FIG. 15, the inner surface 126 of the reaction chamber 120 is a plane, thicknesses of different parts of the first hydrophilic film 181 are different in the direction perpendicular to the inner surface 126, so that the surface of the first hydrophilic membrane 181 away from the inner surface 126 of the reaction chamber 120 is an arc surface, thicknesses of different parts of the second hydrophilic film 182 are different in the direction perpendicular to the inner surface 126, so that the surface of the second hydrophilic film 182 away from the inner surface of the reaction chamber 120 is an arc surface.

FIG. 16 is a cross-sectional schematic diagram of still another microwell array chip provided by an embodiment of the present disclosure. As illustrated by FIG. 16, in the microwell array chip 100, the reaction chamber 120 penetrates through the microwell array substrate 110 in the direction (that is, the direction perpendicular to the first reference plane 201) perpendicular to the microwell array substrate 110. That is, the reaction chamber 120 is a through hole penetrating through the microwell array substrate 110. The inner surface 126 of the reaction chamber 120 is provided with a first hydrophilic film 181 and a second hydrophilic film 182, the first hydrophilic film 181 and the second hydrophilic film 182 are adjacently arranged in the direction perpendicular to the first reference plane 201, the inner side surface 126 of the reaction chamber 120 includes a first sub-surface 1261 and a second sub-surface 1262 in a direction perpendicular to the first reference plane 201, the second sub-surface 1262 is located on a side of the first sub-surface 1261 away from the first main surface 110A, the first sub-surface 1261 protrudes toward the central axis of the reaction chamber 120, so that the surface of the first hydrophilic film 181 away from the inner surface 126 of the reaction chamber 120 is an arc surface, the second sub-surface 1262 protrudes toward the central axis of the reaction chamber 120, so that the surface of the second hydrophilic film 182 away from the inner surface 126 of the reaction chamber 120 is an arc surface. In this way, on the one hand, the first hydrophilic membrane and the second hydrophilic membrane can facilitate that the liquid (such as a sample to be tested) enters into the reaction chamber, and can facilitate the storage of liquid; on the other hand, the surface of the first hydrophilic membrane away from the inner surface of the reaction chamber is an arc surface protruding toward the central axis of the reaction chamber, so that the size of the part of the reaction chamber close to the first main surface is larger, which is more conducive to the liquid entering the reaction chamber; similarly, the surface of the second hydrophilic membrane away from the inner surface of the reaction chamber is arc-shaped, so that the size of the part of the reaction chamber close to the second main surface is larger, which is conducive to gas discharge; at the same time, a recessed part (that is, the adjacent part of the first hydrophilic membrane and the second hydrophilic membrane) that is recessed toward the microwell array substrate can also be formed between the first hydrophilic film and the second hydrophilic film, so that it is more convenient for storing liquid.

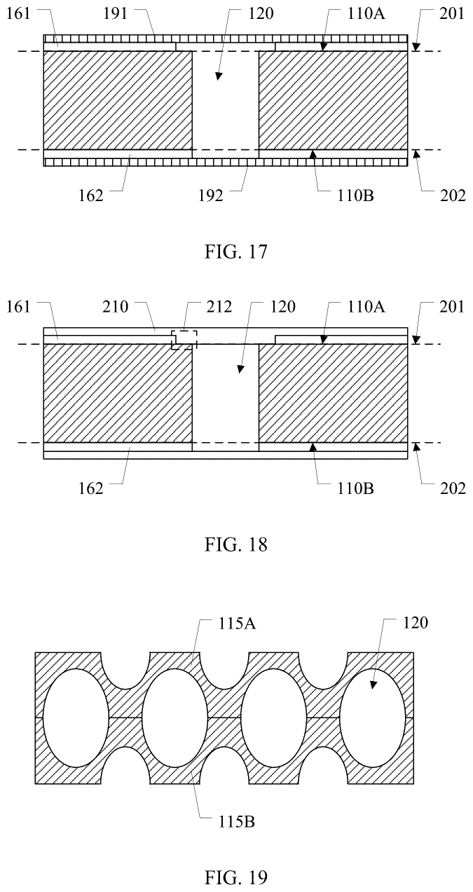

FIG. 17 is a structural schematic diagram of a microwell array chip provided by an embodiment of the present disclosure. As illustrated by FIG. 17, the microwell array chip 100 further includes a first encapsulation film 191 and a second encapsulation film 192; the first encapsulation film 191 is located on a side of the first main surface 110A away from the second main surface 110B; the second encapsulation film 192 is located on a side of the second main surface 110B away from the first main surface 110A; the first encapsulation film 191 and the second encapsulation film 192 are attached to the microwell array substrate 110 by electrostatic adsorption or colloid. In this way, the use of the microwell array chip is relatively convenient, after the liquid (such as the sample to be tested) is introduced into the microwell array substrate, the first encapsulation film and the second encapsulation film can be attached on the microwell array substrate by electrostatic adsorption or colloid.

In some examples, as illustrated by FIG. 17, the microwell array chip 100 further includes a first hydrophobic layer 161 and a second hydrophobic layer 162; the first hydrophobic layer 161 is arranged on the first main surface 110A; an orthographic projection of the first hydrophobic layer 161 on the first reference plane 201 is spaced apart from the orthographic projections of the reaction chambers 120 on the first reference plane 201; and the second hydrophobic layer 162 is located on the second main surface 110B. In this case, the first encapsulation film 191 is located on a side of the first hydrophobic layer 161 away from the second main surface 110B; the second encapsulation film 192 is located on a side of the second hydrophobic layer 162 away from the first main surface 110A. In this way, the first encapsulation film and the second encapsulation film can better encapsulate the microwell array substrate. In addition, the first hydrophobic layer has hydrophobic and lipophilic properties, so that the liquid (such as the sample to be tested) can more easily enter the reaction chambers defined by the microwell array substrate.

For example, a material of the first hydrophobic layer and the second hydrophobic layer may be resin or silicon nitride, for example, epoxy resin. The first hydrophobic layer and the second hydrophobic layer can also be made of other suitable inorganic or organic materials.

FIG. 18 is a cross-sectional schematic diagram of still another microwell array chip provided by an embodiment of the present disclosure; As illustrated by FIG. 18, the microwell array chip 100 also includes photocurable oil 210; the photocurable oil 210 is located at an opening of the reaction chamber 120 close to the first main surface 110A. In this way, the microwell array chip can encapsulate the reaction chamber by the photocurable oil. It should be noted that the photocurable oil can be introduced into a position of the opening of the reaction chamber close to the first main surface, and then the photocurable oil is cured through a light-curing process, so that the reaction chamber is encapsulated.

In some examples, as illustrated by FIG. 18, the microwell array chip 100 further includes a first hydrophobic layer 161, the first hydrophobic layer 161 is arranged on the first main surface 110A; and an orthographic projection of the first hydrophobic layer 161 on the first reference plane 201 is spaced apart from the orthographic projection of the reaction chamber 120 on the first reference plane 201. That is, the first hydrophobic layer 161 does not extend to the edges of the reaction chamber 120, so that it is possible to facilitate the entry of the sample or the reagent into the reaction chamber while preventing the sample or the reagent from remaining in the region outside the reaction chamber.

In some examples, as illustrated by FIG. 18, the photocurable oil 210 includes a convex structure 212, the convex structure 212 is arranged in contact with the first main surface 110A, and is located on a side of the first hydrophobic layer 161 close to the central axis of the reaction chamber 120.

FIG. 19 is a cross-sectional schematic diagram of still another microwell array chip provided by an embodiment of the present disclosure. As illustrated by FIG. 19, the microwell array substrate 110 is a flexible substrate. In this case, the microwell array substrate 110 may include a first sub-flexible microwell array substrate 115A and a second sub-flexible microwell array substrate 115B, the first sub-flexible microwell array substrate 115A and the second sub-flexible microwell array substrate 115B are arranged in contact, and a plurality of reaction chambers 120 are formed between the first sub-flexible microwell array substrate 115A and the second flexible microwell array substrate 115B.

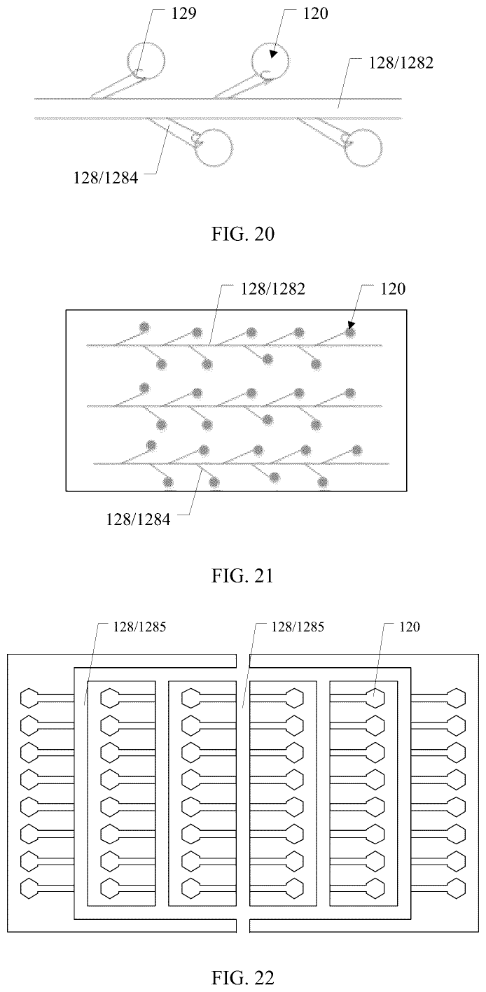

FIG. 20 is a planar schematic diagram of still another microwell array chip provided by an embodiment of the present disclosure. As illustrated by FIG. 20, the microwell array substrate 110 includes a liquid inlet channel 128; the n reaction chambers 120 are in communication with the liquid inlet channel 128, a one-way membrane 129 is arranged between each of the reaction chambers 120 and the liquid inlet channel 128. In this way, the microwell array chip can feed liquid (such as the sample to be tested) into the reaction chamber through the liquid inlet channel, at the same time, the sample to be tested in the reaction chamber can be prevented from returning to the liquid inlet channel through the one-way membrane, so that crosstalk among different reaction chambers can be prevented. Of course, embodiments of the present disclosure include but are not limited thereto, the one-way membrane may not be arranged between each of the reaction chambers and the liquid inlet channel.

In some examples, as illustrated by FIG. 20, the liquid inlet channel 128 includes a liquid inlet main channel 1282 and n liquid inlet branch channels 1284, which communicate with the liquid inlet main channel 1282 respectively; and the n liquid inlet branch channels 1284 and the n reaction chambers 120 are arranged in one-to-one correspondence. In this way, the microwell array chip can feed liquid (such as a sample to be tested) into the reaction chamber through the main liquid inlet channel and the liquid inlet branch channel.

In some examples, as illustrated by FIG. 20, the n liquid inlet branch channels 1284 are arranged on two sides of the liquid inlet main channel 1282, and the plurality of liquid inlet branch channels 1284 located on a first side of the liquid inlet main channel 1282 and the plurality of liquid inlet branch channels 1284 located on a second side of the liquid inlet main channel 1282 are arranged in a dislocation manner.

FIG. 21 is a planar schematic diagram of still another microwell array chip provided by an embodiment of the present disclosure. As illustrated by FIG. 21, the microwell array substrate 110 includes a liquid inlet channel 128; the liquid inlet channel 128 includes a plurality of parallel liquid inlet main channels 1282 and n liquid inlet branch channels 1284; two sides of each of the liquid inlet main channels 1282 are provided with a plurality of liquid inlet branch channels 1284 communicating with the liquid inlet main channel 1282; and the n liquid inlet branch channels 1284 and the n reaction chambers 120 are arranged in one-to-one correspondence. In this way, the microwell array chip can feed liquid (such as a sample to be tested) into the reaction chamber through the liquid inlet channel.

FIG. 22 is a planar schematic diagram of still another microwell array chip provided by an embodiment of the present disclosure. As illustrated by FIG. 22, the microwell array substrate 110 includes a liquid inlet channel 128; the liquid inlet flow channel 128 includes a plurality of sub-liquid inlet flow channels 1285 communicating with each other, and each of the sub-liquid inlet flow channels 1285 communicates with multiple reaction chambers 120. A height of the sub-liquid inlet flow channel 1285 is greater than heights of the multiple reaction chambers 120 connected with the sub-liquid inlet flow channel 1285. In this way, the liquid can flow into the reaction chamber from the sub-liquid inlet flow channel by gravity. It should be noted that the above “height” refers to a distance between the bottom of the sub-liquid inlet flow channel or the reaction chamber and the second main surface of the microwell array substrate.

In some examples, the plurality of sub-liquid inlet flow channels 1285 have different heights. Because the heights of the plurality of sub-liquid inlet flow channels are different, the microwell array chip can make use of gravity to make the liquid (such as the samples to be tested) flow from the higher sub-liquid inlet flow channel to the lower sub-liquid inlet flow channel, so as to enter all the reaction chambers. It should be noted that the above-mentioned “the heights of the plurality of sub-liquid inlet flow channels are different” refers to that distances between the bottoms of the sub-liquid inlet flow channels and the second main surface of the microwell array substrate are different.

In some examples, as illustrated by FIG. 22, a plurality of sub-liquid inlet flow channels 1285 are arranged in sequence, the heights of the plurality of sub-liquid inlet flow channels 1285 decrease sequentially along an arrangement direction of the plurality of sub-liquid inlet flow channels 1285. Of course, embodiments of the present disclosure include but are not limited thereto, the heights of the plurality of liquid inlet channels 1285 may also decrease sequentially from the middle to the two sides.

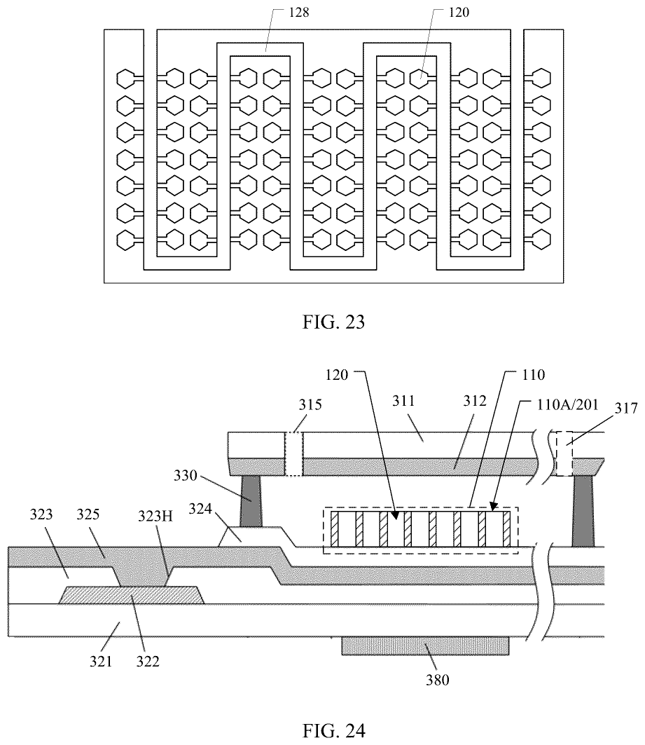

FIG. 23 is a planar schematic diagram of still another microwell array chip provided by an embodiment of the present disclosure. As illustrated by FIG. 23, the microwell array substrate 110 includes a liquid inlet channel 128; a shape of an orthographic projection of the liquid inlet channel 128 on the first reference plane 201 where the first main surface 110A of the microwell array substrate 110 is located is a bent curve. In this way, the microwell array chip can directly feed liquid (such as the samples to be tested) into the reaction chamber through the liquid inlet channel.

FIG. 24 is a cross-sectional schematic diagram of still another microwell array chip provided by an embodiment of the present disclosure. As illustrated by FIG. 24, the microwell array chip 100 further includes a first substrate 310 and a second substrate 320; the first substrate 220 is located on a side of the microwell array substrate 110, and is spaced apart from the first main surface 110A of the microwell array substrate 110; the second substrate 320 is located on a side of the microwell array substrate 110 away from the first substrate 310; the second substrate 320 includes a heating electrode 325, an orthographic projection of the heating electrode 325 on the first reference plane 201 where the first main surface 110A of the microwell array substrate 110 is located is overlapped with the orthographic projections of at least part of the n reaction chambers 120 on the first reference plane 201. In this way, the microwell array chip can heat at least part of the reaction chambers among the n reaction chambers through the heating electrode, so that a heating function is integrated in the microwell array chip.

In some examples, as illustrated by FIG. 24, an orthographic projection of the heating electrode 325 on the first reference plane 201 where the first main surface 110A of the microwell array substrate 110 is located is overlapped with the orthographic projections of the n reaction chambers 120 on the first reference plane 201. In this way, the microwell array chip can simultaneously heat the n reaction chambers through the heating electrodes.

In some examples, as illustrated by FIG. 24, the first substrate 310 includes a first base substrate 311 and a third hydrophobic layer 312; the third hydrophobic layer 312 is located on a side of the first base substrate 311 close to the second substrate 320. In this way, the third hydrophobic layer 312 has hydrophobic and lipophilic properties, so that the liquid (e g, samples to be tested) can be more easily entered into each of the reaction chambers 120 defined by the microwell array substrate 110.

For example, a material of the third hydrophobic layer may be resin or silicon nitride, such as epoxy resin. The third hydrophobic layer can also be made of other suitable inorganic or organic materials, as long as the side of the third hydrophobic layer facing the second substrate is hydrophobic.

In some examples, as illustrated by FIG. 24, the first substrate 310 and the second substrate 310 can form an accommodating space 340 through a sealant 330; the microwell array substrate 110 is arranged in the accommodating space 340; the first substrate 310 also includes at least one sample inlet 315, the sample inlet 315 penetrates through the first base substrate 311 and the third hydrophobic layer 312, and communicates with the accommodating space 340. In this way, the microwell array chip can feed liquid into the microwell array substrate through the sample inlet.

In some examples, as illustrated by FIG. 24, the first substrate 310 further includes at least one sample outlet 317, the sample outlet 317 penetrates through the first substrate 311 and the third hydrophobic layer 312, and communicates with the accommodating space 340. In this way, the microwell array chip can discharge liquid through the sample outlet.

In some examples, as illustrated by FIG. 24, the second substrate 320 further includes a second base substrate 321, a control electrode 322, a first insulating layer 323 and a second insulating layer 324; the control electrode 322 is located on the second base substrate 321; the first insulating layer 323 is located on a side of the control electrode 322 away from the second base substrate 321; the first insulating layer 323 includes a connection hole 323H, and the connection hole 323H exposes at least a part of the control electrode 322; the heating electrode 325 is located on a side of the first insulating layer 323 away from the second base substrate 321, and is connected with the control electrode 322 through the connection hole 323H; the second insulating layer 324 is located on a side of the heating electrode 325 away from the first insulating layer 323, and the microwell array substrate 100 is located on the second insulating layer 324. In this way, the microwell array chip can apply voltage to the heating electrode through the control electrode to drive the heating electrode to generate heat. On the other hand, the second insulating layer is located on a side of the heating electrode away from the first insulating layer, thus the second insulating layer can be used to protect the heating electrode, and prevent the erosion of water and oxygen, so that the service life of the heating electrode is increased. Moreover, the second insulating layer can also play a role of insulation and planarization.

For example, the first insulating layer 323 and the second insulating layer 324 can be made of a same material or different materials. Materials for the first insulating layer 323 and the second insulating layer 324 can be inorganic insulating materials such as silicon oxide, silicon nitride, silicon oxynitride, or organic insulating materials such as resin and polyimide.

In some examples, as illustrated by FIG. 24, the microwell array chip 100 further includes a photosensitive sensor 380; the photosensitive sensor 380 is located on a side of the second substrate 320 away from the first substrate 310, and is configured to detect light emitted by the reaction chamber 120 in the microwell array substrate 110. In this way, the microwell array chip can judge whether a reaction in the reaction chamber occurs and a degree of the reaction through the photosensitive sensor; moreover, the microwell array chip integrates the photosensitive sensor in the chip, so that the degree of integration is further improved.

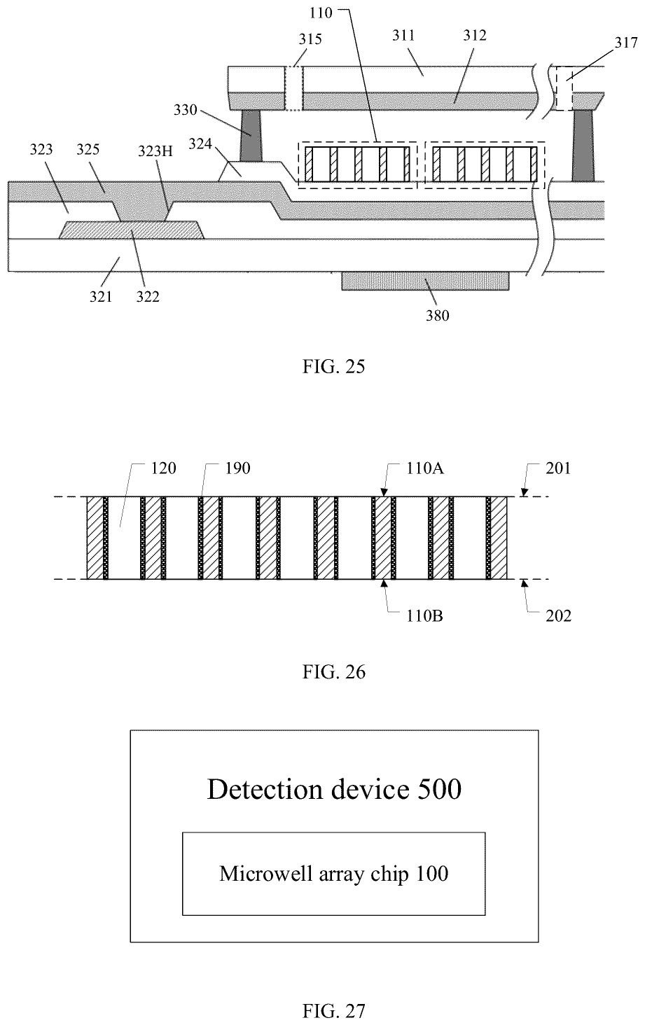

FIG. 25 is a cross-sectional schematic diagram of still another microwell array chip provided by an embodiment of the present disclosure. As illustrated by FIG. 25, the microwell array chip 100 further includes a first substrate 310 and a second substrate 320; the first substrate 220 is located on a side of the microwell array substrate 110, and is spaced apart from the first main surface 110A of the microwell array substrate 110. The microwell array chip 100 includes a plurality of microwell array substrates 110, and are arranged along a direction parallel to the first substrate 310 or the second substrate 320. In this way, the microwell array chip can realize high-throughput detection by arranging a plurality of microwell array chips. It should be noted that, the embodiment of the present disclosure does not specifically limit a planar layout structure of the plurality of microwell array chips. For example, the plurality of microwell array substrates can form a matrix, or the plurality of microwell array substrates can be arranged around one microwell array substrate.

In some examples, as illustrated by FIG. 25, in a case that the microwell array chip includes a plurality of microwell array substrates 110, the second substrate 320 may include a heating electrode 325, an orthographic projection of the heating electrode 325 on the first reference plane 201 where the first main surface 110A of the microwell array substrate 110 is located is overlapped with an orthographic projection of at least part of at least one of the n reaction chambers 120 in the microwell array substrates 110 on the first reference plane 201. In this way, the microwell array chip can heat at least part of the reaction chambers in at least one of the n reaction chambers in the well array substrate through the heating electrodes, so that the heating function is integrated in the microwell array chip. It should be noted that the microwell array chip can also be provided with a plurality of heating electrodes, the plurality of heating electrodes are arranged in one-to-one correspondence with the plurality of microwell array substrates.

In some examples, as illustrated by FIG. 25, the first substrate 310 includes a first base substrate 311 and a third hydrophobic layer 312; the third hydrophobic layer 312 is located on a side of the first base substrate 311 close to the second substrate 320. In this way, the third hydrophobic layer 312 has hydrophobic and lipophilic properties, so that the liquid (such as the sample to be tested) can more easily enter into the reaction chambers 120 defined by the microwell array substrate 110.

For example, the material of the third hydrophobic layer may be resin or silicon nitride, such as epoxy resin. The third hydrophobic layer can also be made of other suitable inorganic or organic materials, as long as the side of the third hydrophobic layer facing the second substrate is hydrophobic.

In some examples, as illustrated by FIG. 25, the first substrate 310 and the second substrate 310 can form an accommodating space 340 through a sealant 330; the microwell array substrate 110 is arranged in the accommodating space 340; the first substrate 310 also includes at least one sample inlet 315, the sample inlet 315 penetrates through the first base substrate 311 and the third hydrophobic layer 312, and communicates with the accommodating space 340. In this way, the microwell array chip can feed liquid into a plurality of microwell array substrates through the injection port.

In some examples, as illustrated by FIG. 25, the first substrate 310 further includes at least one sample outlet 317, the sample outlet 317 penetrates through the first substrate 311 and the third hydrophobic layer 312, and communicates with the accommodating space 340. In this way, the microwell array chip can discharge liquid through the sample outlet.

In some examples, as illustrated by FIG. 25, the second substrate 320 further includes a second base substrate 321, a control electrode 322, a first insulating layer 323, and a second insulating layer 324; the control electrode 322 is located on the second base substrate 321; the first insulating layer 323 is located on a side of the control electrode 322 away from the second base substrate 321; the first insulating layer 323 includes a connection hole 323H, the connection hole 323H exposes at least a part of the control electrode 322; the heating electrode 325 is located on a side of the first insulating layer 323 away from the second base substrate 321, and is connected with the control electrode 322 through the connection hole 323H; the second insulating layer 324 is located on a side of the heating electrode 325 away from the first insulating layer 323, the microwell array substrate 100 is located on the second insulating layer 324. In this way, the microwell array chip can apply voltage to the heating electrode through the control electrode, to drive the heating electrode to generate heat. On the other hand, the second insulating layer is located on a side of the heating electrode away from the first insulating layer, thus the second insulating layer can be used to protect the heating electrode, and prevent the erosion of water and oxygen, so that the service life of the heating electrode is increased. Moreover, the second insulating layer can also play the role of insulation and planarization.

For example, the first insulating layer 323 and the second insulating layer 324 can be made of the same material or different materials. Materials of the first insulating layer 323 and the second insulating layer 324 can be inorganic insulating materials such as silicon oxide, silicon nitride, silicon oxynitride, and organic insulating materials such as resin and polyimide can also be used.

In some examples, as illustrated by FIG. 25, the microwell array chip 100 further includes a photosensitive sensor 380; the photosensitive sensor 380 is located on a side of the second substrate 320 away from the first substrate 310, and is configured to detect light emitted by the reaction chamber 120 in the microwell array substrate 110. In this way, the microwell array chip can judge whether the reaction in the reaction chamber occurs and the degree of the reaction through the photosensitive sensor; moreover, the microwell array chip integrates photosensitive sensors into the chip, so that the degree of integration is further improved.

It is worth noting that the microwell array chip shown in FIG. 25 arranges a plurality of microwell array substrates in a direction parallel to the first substrate or the second substrate, so that high-throughput detection is realized. However, embodiments of the present disclosure include but are not limited thereto, the microwell array chip can also realize high-throughput detection by arranging a plurality of microwell array substrates in the direction perpendicular to the first substrate or the second substrate.

FIG. 26 is a cross-sectional schematic diagram of still another microwell array chip provided by an embodiment of the present disclosure. As illustrated by FIG. 26, the microwell array chip 100 includes a microwell array substrate 110; the microwell array substrate 110 includes a first main surface 110A and a second main surface 110B that are oppositely arranged, n reaction chambers 120 and an idle region 130; the n reaction chambers 120 are arranged in an array in the microwell array substrate 110, and each of the reaction chambers 120 is configured to accommodate a sample to be tested. The microwell array chip 100 also includes an electrohydrophilic changing layer 190, and is arranged on the inner surface 126 of the reaction chamber 120; the electrohydrophilic changing layer 190 is connected with an external circuit through a conductive structure (such as a line), thus the hydrophilicity can be changed (for example, reduce hydrophilicity or change from hydrophilicity to hydrophobicity) by applying electricity, so that the liquid in the reaction chamber 120 can be automatically discharged.

In some examples, the microwell array substrate in the microwell array chip can be made of anti-biological material, so that repeated use can be realized.

In some examples, the microwell array substrate in the microwell array chip can be made of materials that bypass the fluorescence excitation band, so that interference to fluorescence detection is prevented, and detection accuracy is improved.

It should be noted that a total volume V′ of the n reaction chambers 120 in the above-mentioned microwell array chip shown in FIG. 1 to FIG. 26 can satisfy the following formula, to efficiently utilize the area of the microwell array substrate, so that the volume of a single reaction chamber is increased, and the sensitivity of the microwell array chip can be improved, and the detection limit of the microwell array chip is reduced.

$$- 20 n {ln ( 1 - α )}^{\frac{1}{n}} \leq V \leq tan \frac{180 °}{N} \times {N ( \sqrt{\frac{{S}_{chip}}{N tan \frac{{180}^{∘}}{N} ( n + {n}^{′} )}} - X )}^{2} \times n \times h ,$$

In which, (1−α) is the confidence level, S_{chip} is the area of the microwell array substrate 110, h is the depth of the reaction chamber 120 in a direction perpendicular to the first reference plane 201, N is a positive integer greater than or equal to 3, and X is ½ of the size of the interval between adjacent reaction chambers 120. It should be noted that in a case that the value of N is infinite, the above-mentioned regular N-polygon can be a circle.

At least one embodiment of the present disclosure further provides a detection device. FIG. 27 is a schematic diagram of a detection device provided by an embodiment of the present disclosure. As illustrated by FIG. 27, the detection device 500 includes the microwell array chip 100 provided in any of the above examples. Because the microwell array chip can efficiently utilize the area of the microwell array substrate, the volume of a single reaction chamber can be increased, furthermore, the sensitivity of the microwell array chip can be improved, and the detection limit of the microwell array chip can be reduced. In this way, the detection device including the microwell array chip also has higher sensitivity and lower detection limit.

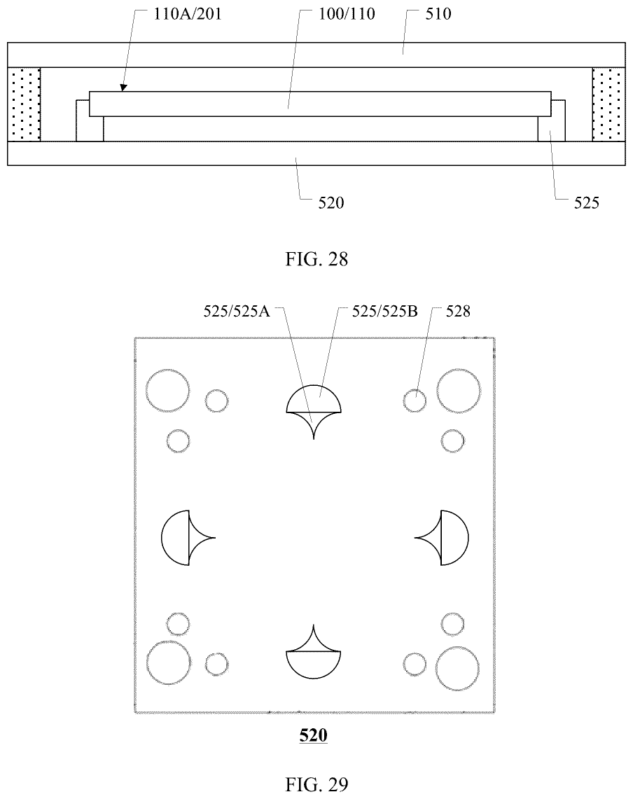

FIG. 28 is a schematic diagram of another detection device provided by an embodiment of the present disclosure. As illustrated by FIG. 28, the detection device 500 further includes a first shell 510 and a second shell 520; the first shell 510 is located on a side of the microwell array chip 100 and is spaced apart from the first main surface 110A; the second shell 520 is located on a side of the microwell array chip 100 away from the first shell 510 and is spaced from the second main surface 110B. The distance between the second main surface 110B and the second shell 520 is greater than or equal to the thickness of the microwell array chip 100. In this way, the detection device can facilitate the entry of liquid into the microwell array chip.

FIG. 29 is a planar schematic diagram of a second shell in a detection device according to an embodiment of the present disclosure. As illustrated by FIG. 28, the second shell 520 includes a support structure 525, the support structure 525 includes a first platform part 525A and a second platform part 525B, the height of the second platform part 525B is greater than the height of the first platform part 525A, the first platform part 525A is configured to be in contact with the bottom surface of the microwell array chip 100, the second platform part 525B is configured to be in contact with the side surface of the microwell array chip 100. In this way, the detection device can support the microwell array chip through the first platform part, and the microwell array chip is positioned through the second platform part, so that the detection accuracy is improved.

In some examples, as illustrated by FIG. 29, a shape of an orthographic projection of the first platform portion 525A on the first reference plane 201 where the first main surface 110A is located includes an arc triangle, a shape of an orthographic projection of the second platform portion 525B on the first reference plane 201 where the first main surface 110A is located (the first reference plane can be seen in FIG. 27) includes a semicircle, a base of the arc triangle connected with the semicircle is a straight line, and the other two sides of the arc triangle are arcs.

In some examples, as illustrated by FIG. 29, the second shell 520 further includes a positioning round table 528, which is configured to be in contact with the side of the microwell array chip 100, so that the microwell array chip is further positioned to improve the detection accuracy.

At least one embodiment of the present disclosure also provides a using method of a microwell array chip. The microwell array chip includes a microwell array substrate; and the using method includes the following steps S101 to S103.

Step S101: introducing a sample to be tested into the microwell array substrate;

Step S102: encapsulating the sample to be tested in a microwell array substrate, in which the microwell array substrate includes a first main surface and a second main surface that are oppositely arranged; the microwell array substrate includes n reaction chambers and an idle region, and the n reaction chambers are arranged in an array in the microwell array substrate and are configured to accommodate samples to be tested; a shape of an orthographic projection of each of the reaction chambers on the first reference plane where the first main surface is located is a regular N-polygon, the idle region is arranged around the n reaction chambers; an area of the idle region is divided into n′ virtual units, a shape of an orthographic projection of each of the virtual units on the first reference plane is the same as the shape of the orthographic projection of each of the reaction chambers on the first reference plane, and the total volume V of the n reaction chambers satisfies the following formula:

$$- 20 n {ln ( 1 - α )}^{\frac{1}{n}} \leq V \leq tan \frac{180 °}{N} \times {N ( \sqrt{\frac{{S}_{chip}}{N tan \frac{{180}^{∘}}{N} ( n + {n}^{′} )}} - X )}^{2} \times n \times h ,$$

In which, (1−α) is the confidence level, S_{chip} is the area of the microwell array substrate, h is the depth of the reaction chamber in the direction perpendicular to the first reference plane, N is a positive integer greater than or equal to 3, X is ½ of a size of the interval between adjacent reaction chambers along a connection line between the centers of the adjacent reaction chambers.

In the using method of the microwell array chip provided by the embodiment of the present disclosure, because the total volume V of the n reaction chambers satisfies the above formula, this method can efficiently utilize the area of the microwell array substrate, so that the volume of a single reaction chamber is increased in a case that the number of reaction chambers is sufficiently large, furthermore, the sensitivity of the microwell array chip can be improved, and the detection limit of the microwell array chip can be reduced.

In some examples, the total volume V of the n reaction chambers satisfies the following formula:

$$15 μ L \leq V \leq tan \frac{180 °}{N} \times N {( \sqrt{\frac{{S}_{chip}}{N tan \frac{{180}^{∘}}{N} ( n + {n}^{′} )}} - X )}^{2} \times n \times h .$$

In this way, the microwell array chip can efficiently utilize the area of the microwell array substrate, so that the sensitivity of the microwell array chip can be improved, and the detection limit of the microwell array chip can be reduced.

In some examples, the microwell array chip further includes a support region and the support region is configured to set a support structure. In this case, the total volume V of the n reaction chambers satisfies the following formula:

$$- 20 n {ln ( 1 - α )}^{\frac{1}{n}} \leq V \leq tan \frac{180 °}{N} \times {N ( \sqrt{\frac{{S}_{chip} - {S}_{support}}{N tan \frac{{180}^{∘}}{N} ( n + {n}^{′} )}} - X )}^{2} \times n \times h ,$$

In which, Sₛᵤₚₚₒᵣₜ is the area of the support region.

In the method of using the microwell array chip provided in this example, the support region can be used to contact the support structure (such as the support structure of the base), so that a certain gap is provided between the microwell array substrate and the base, which is convenient for the sample to be tested to enter, so that the microwell array substrate can be oil-sealed.

In some examples, the shape of the orthographic projection of the reaction chamber on the first reference plane where the first main surface is located and the shape of the orthographic projection of the virtual unit on the first reference plane are all regular hexagons, the total volume V of n reaction chambers satisfies the following formula:

$$- 20 n {ln ( 1 - α )}^{\frac{1}{n}} \leq V \leq tan 30 ° \times 6 {( \sqrt{\frac{{S}_{chip}}{N tan 30 ° - ( n + {n}^{′} )}} - X )}^{2} \times n \times h ,$$

In which, the value range of X is from 10 microns to 20 microns, and the value range of h is from 190 microns to 320 microns.

In the microwell array chip provided in the example, the shape of the orthographic projection of each of the reaction chambers on the first reference plane is a regular hexagon, and because the total volume V of the n reaction chambers satisfies the above formula, the microwell array chip can efficiently utilize the area of the microwell array substrate, so that the volume of a single reaction chamber is increased, furthermore, the sensitivity of the microwell array chip can be improved, and the detection limit of the microwell array chip can be reduced.

In some examples, the distance between adjacent reaction chambers may be in the range from 20 microns to 40 microns on the line between the centers of adjacent reaction chambers, for example, 24 microns, 26 microns, 28 microns, 30 microns, 32 microns, 34 microns or 36 microns.

In some examples, the depth of each of the reaction chambers in the direction perpendicular to the first reference plane may be 200 microns, 220 microns, 240 microns, 260 microns, 280 microns, or 300 microns.

In some examples, the number of the reaction chambers on one microwell array substrate may be from 8,000 to 100,000. In this way, the microwell array chip has higher detection accuracy.

In some examples, the number of the reaction chambers on one microwell array substrate can be 8,000, 10,000, 20,000, 40,000, 60,000, 80,000, or 100,000.

In some examples, encapsulating the sample to be tested in the microwell array substrate includes: after introducing the sample to be tested into the microwell array substrate, attaching the first encapsulation film on a side of the first main surface away from the second main surface by static electricity or colloid, and attaching the second encapsulation film on a side of the second main surface away from the first main surface. In this way, the microwell array chip can simply package the microwell array substrate, and the efficiency is high.

In some examples, encapsulating the sample to be tested in a microwell array substrate includes: after the sample to be tested is introduced into the microwell array substrate, coating photocurable oil at the opening position of the reaction chamber close to the first main surface; and curing the photocurable oil with ultraviolet light. In this way, the microwell array chip can use photocurable oil and photocurable technology to encapsulate the microwell array substrate, and has the advantages of simplicity, high efficiency and good packaging effect.

In some examples, in a case that the flexible substrate shown in FIG. 19 is used as the microwell array substrate, the microwell array substrate may include a first sub-flexible microwell array substrate and a second sub-flexible microwell array substrate, and a plurality of sample flow channels are included between the first sub-flexible microwell array substrate and the second sub-flexible microwell array substrate. Introducing the sample to be tested in the microwell array substrate includes: introducing the sample to be tested in a plurality of sample flow channels.

For example, the sample to be tested can be passed into the plurality of sample flow channels by refers to of vacuum adsorption and pumping.

In some examples, encapsulating the sample to be tested in a microwell array substrate includes: using rollers to separate each of the sample flow channels to form a plurality of reaction chambers, and sealing the plurality of reaction chambers. For example, the roller has a heating function, and the first sub-flexible microwell array substrate and the second flexible microwell array substrate are combined by heating at a certain distance, so that each of the sample flow channels can be divided to form a plurality of reaction chambers.

The following points required to be explained:
(1) the drawings of the embodiments of the present disclosure only relate to the structures related to the embodiments of the present disclosure, and other structures can refer to the general design.
(2) without conflict, the embodiments of the present disclosure and the features in the embodiments may be combined with each other.

The above is only the specific embodiment of this disclosure, but the protection scope of the present disclosure is not limited thereto. Any person familiar with the technical field can easily think of changes or substitutions within the technical scope disclosed in the present disclosure, and they should be included in the protection scope of the present disclosure. Therefore, the scope of protection of the present disclosure should be based on the scope of protection of the claims.

1. A microwell array chip, comprising:
a microwell array substrate, comprising a first main surface and a second main surface that are oppositely arranged;
n reaction chambers, wherein the n reaction chambers are arranged in an array in the microwell array substrate, and are configured to accommodate a sample to be tested, and a shape of an orthographic projection of each of the reaction chambers on a first reference plane where the first main surface is located is a regular N-polygon; and
an idle region, arranged around the n reaction chambers;
wherein an area of the idle region is divided into n′ virtual units, a shape of an orthographic projection of each of the virtual units on the first reference plane is the same as the shape of the orthographic projection of each of the reaction chambers on the first reference plane, a total volume V of the n reaction chambers satisfies the following formula:
$$- 20 n {ln ( 1 - α )}^{\frac{1}{n}} \leq V \leq tan \frac{180 °}{N} \times {N ( \sqrt{\frac{{S}_{chip}}{N tan \frac{{180}^{∘}}{N} ( n + {n}^{′} )}} - X )}^{2} \times n \times h ,$$
wherein (1−α) is a confidence level, S_{chip} is an area of the microwell array substrate, h is a depth of each of the reaction chambers in a direction perpendicular to the first reference plane, N is a positive integer greater than or equal to 3, and X is ½ of a size of an interval between adjacent reaction chambers along a connection line between centers of the adjacent reaction chambers.

2. The microwell array chip according to claim 1, further comprising:
a support region, configured to set a support structure,
wherein the total volume V of the n reaction chambers satisfies the following formula:
$$- 20 n {ln ( 1 - α )}^{\frac{1}{n}} \leq V \leq tan \frac{180 °}{N} \times {N ( \sqrt{\frac{{S}_{chip} - {S}_{support}}{N tan \frac{{180}^{∘}}{N} ( n + {n}^{′} )}} - X )}^{2} \times n \times h ,$$
wherein, Sₛᵤₚₚₒᵣₜ is an area of the support region.

3. The microwell array chip according to claim 2, further comprising:
a reaction region, arranged on a periphery of the support region,
wherein the n reaction chambers are located in the reaction region.

4. The microwell array chip according to claim 3, wherein, in the reaction region, distances between centers of orthographic projections of two adjacent reaction chambers on the first reference plane are equal.

5. The microwell array chip according to claim 1, wherein a shape of the orthographic projection of the reaction chamber on the first reference plane where the first main surface is located is a regular hexagon, the total volume V of the n reaction chambers satisfies the following formula:
$$- 20 n {ln ( 1 - α )}^{\frac{1}{n}} \leq V \leq tan {30}^{∘} \times 6 {( \sqrt{\frac{{S}_{c h i p}}{N tan {30}^{∘} ( n + {n}^{′} )}} - X )}^{2} \times n \times h ,$$
wherein a value range of X is from 10 microns to 20 microns, and a value range of h is from 190 microns to 320 microns.

6. The microwell array chip according to claim 1, wherein a value range of n is from 8000 to 100000.

7. The microwell array chip according to claim 1, wherein the total volume V of the n reaction chambers satisfies the following formula:
$$15 μ L \leq V \leq tan \frac{180 °}{N} \times N {( \sqrt{\frac{{S}_{chip}}{N tan \frac{{180}^{∘}}{N} ( n + {n}^{′} )}} - X )}^{2} \times n \times h .$$

8. The microwell array chip according to claim 1, further comprising:
a first hydrophobic layer, arranged on the first main surface,
wherein an orthographic projection of the first hydrophobic layer on the first reference plane is spaced apart from the orthographic projections of the reaction chambers on the first reference plane.

9. (canceled)

10. The microwell array chip according to claim 8, further comprising:
a second hydrophobic layer, arranged on the second main surface,
wherein the second hydrophobic layer extends to edges of the reaction chambers, or the reaction chambers penetrate through the microwell array substrate in a direction perpendicular to the first reference plane, the second hydrophobic layer spans across the reaction chambers, and orthographic projections of the reaction chambers on a second reference plane where the second main surface is located fall within an orthographic projection of the second hydrophobic layer on the second reference plane.

11. (canceled)

12. (canceled)

13. (canceled)

14. The microwell array chip according to claim 8, wherein each of the reaction chambers is recessed into the microwell array substrate from the first main surface, and has a chamber bottom located in the microwell array substrate, and a distance between the chamber bottom and the first reference plane is smaller than the thickness of the microwell array substrate,
a contact angle between the first hydrophobic layer and the sample to be tested is smaller than a critical angle of the reaction chamber, the critical angle is an angle between an extension line of a side wall of the reaction chamber and a tangent line of a surface of the sample to be tested which is in contact with the side wall of the reaction chamber.

15. (canceled)

16. The microwell array chip according to claim 14, wherein the chamber bottom comprises at least one exhaust hole, each of the at least one exhaust hole penetrates through the chamber bottom in a direction perpendicular to the first reference plane.

17. (canceled)

18. (canceled)

19. The microwell array chip according to claim 8, further comprising:
a dialysis membrane; and
a second hydrophobic layer,
wherein the reaction chambers penetrate through the microwell array substrate in a direction perpendicular to the first reference plane, the second hydrophobic layer extends to edges of the reaction chambers, and the dialysis membrane spans across one reaction chamber.

20. (canceled)

21. (canceled)

22. (canceled)

23. (canceled)

24. The microwell array chip according to claim 1, wherein an included angle between an inner surface of each of the reaction chambers and the first main surface is greater than 90 degrees,
or an inner side surface of each of the reaction chambers comprises a first sub-surface and a second sub-surface in a direction perpendicular to the first reference plane, the second sub-surface is located on a side of the first sub-surface away from the first main surface, an angle between the first sub-surface and the first main surface is greater than 90 degrees, and an included angle between the second sub-surface and the second main surface is greater than 90 degrees,
or an inner side surface of each of the reaction chambers comprises a first sub-surface, a second sub-surface, and a third sub-surface in a direction perpendicular to the first reference plane, and the second sub-surface is located on a side of the first sub-surface away from the first main surface, and the third sub-surface is located on a side of the second sub-surface away from the first sub-surface, and an included angle between the first sub-surface and the first main surface is greater than 90 degrees, a plane where the second sub-surface is located is perpendicular to the first reference plane, and an included angle between the third sub-surface and the second main surface is greater than 90 degrees,
or, an inner side surface of each of the reaction chambers comprises a first sub-surface, a second sub-surface, and a third sub-surface in a direction perpendicular to the first reference plane, the second sub-surface is located on a side of the first sub-surface away from the first main surface, and the third sub-surface is located on a side of the second sub-surface away from the first sub-surface, and an included angle between the first sub-surface and the first main surface is greater than 90 degrees, the second sub-surface is an arc surface, and is recessed toward the microwell array substrate, and an included angle between the third sub-surface and the second main surface is greater than 90 degrees.

25. (canceled)

26. (canceled)

27. (canceled)

28. The microwell array chip according to claim 1, wherein an inner surface of each of the reaction chambers is provided with a first hydrophilic membrane and a second hydrophilic membrane, the first hydrophilic membrane and the second hydrophilic membrane are adjacently arranged in a direction perpendicular to the first reference plane, a surface of the first hydrophilic film away from the inner surface of each of the reaction chambers is an arc surface protruding toward a central axis of the reaction chamber, and a surface of the second hydrophilic film away from the inner surface of each of the reaction chambers is an arc surface protruding toward a central axis of each of the reaction chambers.

29. (canceled)

30. (canceled)

31. The microwell array chip according to claim 1, wherein a shape of an orthographic projection of each of the reaction chambers on the first reference plane comprises one selected from the group consisting of a circle, a regular hexagon, a regular octagon, and a triangle.

32. (canceled)

33. (canceled)

34. (canceled)

35. (canceled)

36. The microwell array chip according to claim 1, wherein the microwell array substrate further comprises:
a liquid-inlet channel,
the n reaction chambers are in communication with the liquid-inlet channel, and a one-way membrane is arranged between each of the reaction chambers and the liquid-inlet channel.

37. (canceled)

38. (canceled)

39. (canceled)

40. (canceled)

41. The microwell array chip according to claim 1, further comprising:
a first substrate, located on a side of the microwell array substrate, and spaced apart from the first main surface; and
a second substrate, located on a side of the microwell array substrate away from the first substrate,
wherein the second substrate comprises a heating electrode, an orthographic projection of the heating electrode on the first reference plane is overlapped with orthographic projections of at least part of the n reaction chambers on the first reference plane.

42. (canceled)

43. (canceled)

44. (canceled)

45. A detection device, comprising the microwell array chip according to claim 1.

46. The detection device according to claim 45, further comprising:
a first shell, located on a side of the microwell array chip, and spaced apart from the microwell array chip; and
a second shell, located on a side of the microwell array chip away from the first shell, and spaced apart from the microwell array chip,
wherein a distance between the microwell array chip and the second shell is greater than or equal to the thickness of the microwell array chip,
the second shell comprises a support structure comprising a first platform part and a second platform part, a height of the second platform part is greater than a height of the first platform part, the first platform part is configured to be in contact with a bottom surface of the microwell array chip, and the second platform part is configured to be in contact with a side surface of the microwell array chip,
a shape of an orthographic projection of the first platform part on the first main surface comprises an arc triangle, a shape of an orthographic projection of the second platform part on the first main surface comprises a semicircle, a bottom of the arc triangle connected with the semicircle is a straight line, and the other two sides of the arc triangle are arcs,
the second shell further comprises a positioning circular platform, and the positioning circular platform is configured to be in contact with a side surface of the microwell array chip.

47. (canceled)

48. (canceled)

49. (canceled)

50. A using method of a microwell array chip, wherein the microwell array chip comprises a microwell array substrate, the using method of a microwell array chip comprises:
introducing a sample to be tested into the microwell array substrate;
encapsulating the sample to be tested in the microwell array substrate,
wherein the microwell array substrate includes n reaction chambers and a virtual idle region, the n reaction chamber are arranged in an array in the microwell array substrate, and is configured to accommodate the samples to be tested; a shape of an orthographic projection of each of the reaction chambers on the first reference plane where the first main surface is located is a regular N-polygon, the virtual idle region is arranged around the n reaction chambers; an area of the virtual idle region is divided into n′ virtual units, a shape of an orthographic projection of each of the virtual units on the first reference plane is the same as the shape of the orthographic projection of each of the n reaction chambers on the first reference plane, a total volume V of the n reaction chambers satisfies the following formula:
$$- 20 n {ln ( 1 - α )}^{\frac{1}{n}} \leq V \leq tan \frac{18 {0}^{∘}}{N} \times {N ( \sqrt{\frac{{S}_{c h i p}}{N tan \frac{18 {0}^{∘}}{N} ( n + {n}^{′} )}} - X )}^{2} \times n \times h ,$$
wherein (1−α) is the confidence level, S_{chip} is an area of the microwell array substrate, h is the depth of the reaction chamber in a direction perpendicular to the first reference plane, N is a positive integer greater than or equal to 3, and X is ½ of a size of the interval between adjacent reaction chambers at a connection line between the centers of the adjacent reaction chambers.

51. (canceled)

52. (canceled)

53. (canceled)

54. (canceled)

55. (canceled)
